(19) United States
(12) Patent Application Publication
(10) Pub. No.: US 2018/0133148 A1
(43) Pub. Date: May 17, 2018

(54) Degradable Microparticles for Protein and Small Molecule Delivery

(71) Applicant: Georgia Tech Research Corporation, Atlanta, GA (US)

(72) Inventors: Liane Elizabeth Tellier, Atlanta, GA (US); Edward Botchwey, Atlanta, GA (US); Alexandra Ann Falis, Atlanta, GA (US); Johnna Sue Temenoff, Atlanta, GA (US)

(21) Appl. No.: 15/782,388

(22) Filed: Oct. 12, 2017

Related U.S. Application Data
(60) Provisional application No. 62/408,224, filed on Oct. 14, 2016.

(51) Int. Cl. A61K 9/00; A61K 9/14; A61K 38/19; A61P 21/00; A61P 19/04

(52) U.S. Cl. CPC A61K 9/0024; A61K 9/146; A61K 38/191; A61P 21/00; A61P 19/04; A61K 38/195

(57) ABSTRACT

The invention relates to a microparticle platform and pharmaceutical compositions capable of recruiting pro-regenerative cells to and/or reducing inflammation at the site of muscle, joint, tendon, and/or ligament damage. The invention further relates to methods of treating muscle, joint, tendon, and/or ligament damage comprising administering the microparticles or pharmaceutical compositions comprising the microparticles.

### CROSS-REFERENCE TO RELATED APPLICATIONS

This application claims priority to U.S. Provisional Application Ser. No. 62/408,224, filed on Oct. 14, 2016, the contents of which are incorporated herein by reference in their entirety.

### STATEMENT REGARDING FEDERALLY SPONSORED RESEARCH

This invention was made with United States Government support under National Institute of Health (NIH) Grant No. R01 AR063692. The United States Government has certain rights to this invention.

### FIELD OF INVENTION

The present invention relates to a microparticle platform capable of recruiting pro-regenerative cells and/or reducing inflammation at the site of muscle, joint, tendon, or ligament damage.

### BACKGROUND

There is a need to provide improved treatment of muscle injuries. For example, rotator cuff tears affect up to 40% of the general population and the prevalence of rotator cuff injury increases with age. Yamaguchi K, Ditsios K, Middleton W D, Hildebolt C F, Galatz L M, Teefey S A. J Bone Jt Surg. 2006; 88(8):1699-1704. Current standard-of-care reattachment surgery largely neglects the muscle tissue, and ultimately muscle degeneration will either remain the same or advance after reattachment of the tendon. Gladstone J N, Bishop J Y, Lo I K Y, Flatow E L. Am J Sports Med. 2007: 719-728; Gerber C, Fuchs B, Hodler J. J Bone Joint Surg Am. 2000; 82(4):505-515. This can be problematic since the severity of muscle degeneration following rotator cuff tear has shown to positively correlate with re-tear after treatment. Therefore, there is a strong need in the field for a strategy to prevent or reverse muscle degeneration in order to improve overall clinical repair outcomes following rotator cuff tear or any other type of muscle injury.

Following skeletal muscle injury, regeneration conventionally involves two inter-dependent periods: a degenerative inflammatory phase followed by a reparative phase. Chargé SBP, Rudnicki M. Physiol Rev. 2004; 84(1):209-238. During the degenerative phase, leukocyte subsets such as neutrophils typically respond within the first 48 hours, monocytes infiltrate within 5 hours, and monocyte-derived macrophages become the dominant cell type in days to weeks. Karalaki M, Fili S, Philippou A, Koutsilieris M. In Vivo. 2009; 23(5):779-796; Roche J A, Tulapurkar M E, Mueller A L, et al. Am J Pathol. 2015; 185(6):1686-1698.

Though a spectrum of phenotypes exist, macrophages are often categorized as classically activated, pro-inflammatory M1 and alternatively activated, anti-inflammatory M2 macrophages. Ogle M E, Segar C E, Sridhar S, Botchwey E A. Exp Biol Med. 2016; 241(10):1084-1097; Tidball J G. Am J Physiol—Integr Comp Physiol. 2005; 288:345-353. M1 macrophages have been shown to infiltrate quickly following muscle injury and act to phagocytose cellular debris and secrete pro-inflammatory proteins such as tumor necrosis factor-alpha (TNF-α) and interferon-gamma (IFN-γ). As early as one day following injury, however, macrophages can transition toward a more anti-inflammatory M2 phenotype, which becomes the dominate macrophage population by day 4 post-injury. Chazaud B, Brigitte M, Yacoub-Youssef H, et al. Exerc Sport Sci Rev. 2009; 37(1):18-22. M2 macrophages not only secrete anti-inflammatory proteins such as transforming growth factor-beta (TGF-β), interleukin-10 (IL-10), and insulin-like growth factor-1 (IGF-1), but have also shown to activate and enhance the proliferation and differentiation of satellite cells, a muscle-derived stem cell subpopulation and the primary contributor to the reparative phase of muscle regeneration. Merly F, Lescaudron L, Rouaud T, Crossin F, Marie France Gardahaut. Muscle Nerve. 1999; (June):724-732; Peng H, Huard J. Transpl Immunol. 2004; 12(3-4):311-319. Subsequently, macrophages have also been shown to enhance myoblast proliferation and differentiation in vitro and muscle membrane repair and fiber growth in vivo. Arnold L, Henry A, Poron F, et al. J Exp Med. 2007; 204(5):1057-1069; Massimino M L, Rapizzi E, Cantini M, et al. Biochem Biophys Res Commun. 1997; 235(3):754-759; Deng B, Wehling-Henricks M, Villalta S A, Wang Y, Tidball J G. J Immunol. 2012; 189(7):3669-3680; Tidball J G, Wehling-henricks M. J Physiol. 2007; 1:327-336.

Unlike other muscle injuries, in a rotator cuff tendon tear the cellular mechanisms for muscle healing become dysregulated or dysfunctional, resulting in inadequate muscle regeneration. Meyer G A, Farris A L, Sato E, et al. J Orthop Res. 2015; (March):421-429; Thomas K A, Gibbons M C, Lane J G, Singh A, Ward S R, Engler A J. J Orthop Res. 2016: 12-14. Thus, recruitment of endogenous pro-regenerative cell populations from the bone marrow to the muscle following rotator cuff injury may be critical to improve muscle healing. The chemotactic protein stromal cell-derived factor-1alpha (SDF-1α), primarily through its G protein-coupled receptor, CXC chemokine receptor type 4 (CXCR4), has previously shown to attract an array of pro-regenerative, CXCR4-expressing cell populations including inflammatory cells, mesenchymal stem cells (MSCs), and hematopoietic stem cells (HSCs), among others. Otsuru S, Tamai K, Yamazaki T, Yishikawa H, Kaneda Y. Stem Cells. 2008; 26:223-234; Krieger J R, Ogle M E, McFaline-Figueroa J, Segar C E, Temenoff J S, Botchwey E A. Biomaterials. 2015; Kucia M, Jankowski K, Reca R, et al. J Mol Histol. 2004: 233-245. Following myocardial infarction (MI) in mice, for example, the endogenous upregulation of SDF-1 mRNA and protein expression was observed and led to an ˜80% increase in the homing of bone marrow-derived cells compared to uninjured controls. Abbott J D, Huang Y, Liu D, et al. Coron Hear Dis. 2004; 110:3300-3305. In other studies, SDF-1-overexpressing cells were transplanted after myocardial infarction, which induced significantly more homing of CD117+ stem cells compared to uninjured controls. Askari A T, Unzek S, Popovic Z B, et al. Lancet. 2003; 362:697-703.

An experiment in which SDF-1α loaded hydrogels were subcutaneously implanted into a murine dorsal skinfold window chamber model, a model which enables the visualization of cell recruitment and vascular remodeling showed that more bone marrow-derived cells and significantly more anti-inflammatory monocytes were detected and after 7 days. Also, significantly more M2-like macrophages were observed near SDF-1α loaded gels as compared to unloaded controls. Ogle M E, Krieger J R, Tellier L E, McFaline-Figueroa J, Temenoff J S, Botchwey E A. ACS Biomater Sci Eng. 2017: acsbiomaterials.6b00706.

SDF-1α delivery has not yet been investigated in the context of rotator cuff injury.

Natively sulfated heparin possesses potent anti-coagulant properties and may present safety issues in vivo. Biomaterials containing heparin, a highly sulfated glycosaminoglycan (GAG) that can bind and interact with a myriad of proteins to maintain or enhance protein bioactivity, including SDF-1α, have been developed. Tellier L E, Miller T, McDevitt T C, Temenoff J S. J Mater Chem B. 2015; Peng Y, Tellier L E, Temenoff J S. Biomater Sci. 2016; 4(9):1371-1380; Sadir R, Imberty A, Lortat-jacob H. J Biol Chem. 2004; 279(42):43854-43860; and Purcell B P, Elser J A, Mu A, Margulies K B, Burdick J A. Biomaterials. 2012; 33(31):7849-7857. Heparin and heparin derivatives have been successfully incorporated within bulk hydrogels for SDF-1α delivery. Prokoph S, Chavakis E, Levental K R, et al; Biomaterials. 2012; 33(19):4792-4800; Anderson E M, Kwee B J, Lewin S A, Raimondo T, Mehta M, Mooney D J. Tissue Eng Part A. 2015; 21(7-8):1217-1227.

There is a need for novel forms of delivery of SDF-1α, and any other proteins that promote muscle healing via cell recruitment, to injured muscle tissue. In addition, there is a need to localize SDF-1α and other proteins that promote muscle healing to the muscle for a period of time so that such proteins can recruit cells to the muscle that play a role in healing injured muscle tissue.

There is also a need to provide improved treatment of injuries and damage to joints, tendons and ligaments. Osteoarthritis (OA) affects more than 60% of Americans over 65 years of age and is characterized by significant articular cartilage degeneration including cartilage fibrillation, fissures, and loss of proteoglycan and collagen content within the cartilage extracellular matrix (ECM). Gerwin N, Hops C, Lucke A. Adv Drug Deliv Rev. 2006; 58(2):226-242; Willett N J, Thote T, Lin A S, et al. Arthritis Res Ther. 2014; 16(1):R47; Goldriing M B. Curr Rheumatol Rep. 2000; 2(6):459-465. While OA is a multi-factorial disease, at stages of disease progression both cartilage-resident chondrocytes and synoviocytes in the joint capsule have been found to secrete increased levels of soluble factors including interleukins, tumor necrosis factor-alpha (TNF-α), and matrix metalloproteinases (MMPs), among others, which are known to promote or be directly involved in cartilage degradation. Pearle A D, Warren R F, Rodeo S A. Clin Sports Med. 2005; 24(1):1-12; Abramson S B. J Rheumatol. 2004; 31(SUPPL. 70):70-76. Additionally, components of the plasminogen activation pathway including plasminogen and plasminogen activators, receptors, and inhibitors have also been shown to be upregulated in OA joints when compared to healthy patients. Kummer J A, Abbink J J, Boer J D E, et al. Arthritis Rheum. 1992; 35(8); Brommer P, Dooijewaard G, Dijkmans B, Breedvelf F. Ann Rheum Dis. 1992; 51:965-968; Belcher C, Fawthrop F, Bunning R, Doherty M. Ann Rheum Dis. 1996; 55:230-236. As plasmin, the active form of plasminogen, has been shown to activate MMPs and degrade ECM, it is postulated that the plasminogen activation pathway plays a significant role in the cartilage degeneration exhibited in OA joints. Ronday H K, Smits H H, Van Muijen G N, et al. Br J Rheumatol. 1996; 35(5):416-423; Wisniewski H G, Vilčk J. Cytokine Growth Factor Rev. 1997; 8(2):143-156; Wisniewski H G, Hua J C, Poppers D M, Naime D, Vilcek J, Cronstein B N. J Immunol. 1996; 156:1609-1615. Thus, plasmin-inhibiting therapeutics may be a promising method to ameliorate cartilage degeneration in the context of OA.

TNF-α-stimulated gene-6 (TSG-6) is a positively charged 35 kDa protein with anti-plasmin and anti-inflammatory properties. In particular, TSG-6 has been studied extensively for its ability to potentiate inter-alpha-inhibitor (IalphaI)-mediated inhibition of plasmin and more recently, its ability to suppress the response of chondrocytes to inflammatory factors such as interleukin-I and TNF-α. Day A J, Drummond S P, Anand S, Bartnik E, Milner C M. Osteoarthr Cartil. 2016; 24(2016):S19-S20. In the context of OA, while little constitutively expressed TSG-6 has been observed in healthy patients, TSG-6 protein expression was found to be upregulated in OA joints and greater TSG-6 levels were observed in patients where OA symptoms had advanced over a three year period compared to non-progressing OA patients. Bayliss M T, Howat S L T, Dudhia J, et al. Osteoarthr Cartil. 2001; 9(1):42-48; Wisniewski H, Colón E, Liublinska V, et al. Osteoarthr Cartil. 2014; 22(2):235-241; Chou C, Wisniewski H, Band P, et al. Osteoarthr Carta. 2016; 24(2016):S81-S82.

Despite the increased production of endogenous TSG-6, however, tissue within OA joints continues to degenerate, leading to studies on the effect of adding exogenous TSG-6 on arthritis progression. In rheumatoid arthritis (RA) mouse models, soluble TSG-6 treatment led to a significant improvement in joint swelling and cartilage damage, assessed via joint diameter and histology, respectively, but these effects were often short-lived, and by day 12 or day 35 no differences were observed between treated and untreated animals. Bárdos T, Kamath R V., Mikecz K, Glant T T. Am J Pathol. 2001; 159(5):1711-1721. doi:10.1016/S0002-9440(10)63018-0; Glant T T, Kamath R V., Bárdos T, et al. Arthritis Rheum. 2002; 46(8):2207-2218. Most recently, soluble TSG-6 treatment was also investigated in an OA model induced by anterior cruciate ligament and meniscus transection. In this work, a portion of the TSG-6 molecule was delivered via intra-articular injection weekly up to 21 days following injury. After 28 days, histology indicated that cartilage fibrillation and ulceration were significantly diminished with TSG-6 treatment compared to untreated controls, indicating that TSG-6 or TSG-6 derivatives may be an effective OA treatment strategy.

Despite these promising results, one drawback to soluble treatments are often the high doses required due to rapid clearance from the joint space. Owen S G, Francis H W, Roberts M S. Br J Clin Pharmacol. 1994: 349-355. Therefore, there is a strong unmet need in the field to develop new treatments of OA that overcome the drawbacks and challenges outlined above.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF THE INVENTION

Various non-limiting aspects and embodiments of the invention are described below.

In one aspect, the present invention describes microparticles (MPs) comprising a protein and/or a small molecule active agent. One exemplary small molecule is the sphingosine analog FTY720. The small molecule agent and protein may be delivered together on one bifunctional MP.

In one embodiment, the MPs of the invention may be carbohydrate-based MPs, e.g., glycosaminoglycan (GAG)-based MPs, e.g., heparin-based MPs, i.e., GAG, such as N-desulfated heparin (Hep^{-N}) may be incorporated within the microparticles of the invention. In another embodiment, dithiothreitol (DTT) may be incorporated within the MPs. In another embodiment, poly (ethylene glycol)-based linker, e.g., poly (ethylene glycol) diacrylate (PEGDA)-based linker may be utilized to incorporate DTT into GAG-based MPs. In one embodiment, the MPs may incorporate a protein and/or a small molecule active agent. In one embodiment, the protein incorporated within the MPs of the invention may comprise SDF-1α. In another embodiment, the protein incorporated within the MPs of the invention may comprise TSG-6. In another embodiment, the small molecule incorporated within the MPs may comprise FTY720. In another embodiment, a protein, e.g., SDF-1α, and a small molecule, e.g., FTY720, may simultaneously be incorporated within one bifunctional MP. In one embodiment, the microparticles of the invention may be between about 10 and about 500 micrometers in diameter, or between about 20 and about 100 micrometers in diameter, or between about 30 and about 80 micrometers in diameter.

In another aspect, pharmaceutical compositions comprising MPs of the invention are described. In one embodiment, pharmaceutical compositions of the invention comprise heparin-based MPs comprising a protein and/or a small molecule (e.g., the sphingosine analog FTY720) and a pharmaceutically acceptable carrier, additive, or excipient.

In another aspect, pharmaceutical dosage forms comprising MPs of the invention are described. In various embodiments, pharmaceutical dosage forms of the invention may be liquid, semi-solid, powder, or solid dosage forms. In one embodiment, pharmaceutical dosage forms of the invention are liquid injectable dosage forms. The liquid injectable dosage forms may comprise one or more of an excipient, a carrier, a buffer, and a salt.

In another aspect, the present invention describes a method of fabricating MPs of the invention, said method comprising forming a water-and-oil emulsion. In some embodiments, fabricating a degradable Hep^{-N} microparticle comprises adding PEGDA and dithiothreitol (DTT) to a solution comprising bovine serum albumin (BSA) in PBS, incubating at 30-40° C. for 20-40 minutes, adding Hep^{-N}MAm to the solution, and incubating the aqueous phase for 20-40 minutes.

In another aspect, the present invention describes a method of recruiting pro-regenerative cells to, and/or reducing inflammation at, the site of muscle, joint, tendon, and/or ligament damage. In this aspect, the method of the invention comprises introducing MPs of the invention comprising an effective amount of a protein and/or a small molecule active agent (e.g., the sphingosine analog FTY720) into the area of muscle, tendon, ligament or joint damage by, e.g., an intramuscular or intra-articular injection. In some embodiments, pharmaceutical compositions comprising MPs of the invention comprising an effective amount of a protein and/or a small molecule active agent are introduced into the area of muscle, tendon, ligament or joint damage by an intramuscular or intra-articular injection to recruit pro-regenerative cells to, and/or reduce inflammation at, the site of muscle, tendon, ligament or joint damage. In other embodiments, a pharmaceutical dosage form comprising MPs of the invention may be utilized to deliver MPs of the invention to the site of muscle, tendon, ligament or joint damage to recruit pro-regenerative cells to, and/or reduce inflammation at, the site of muscle or joint damage.

In another aspect, the present invention describes a method of treating muscle or joint damage by introducing MPs of the invention comprising an effective amount of a protein and/or a small molecule active agent into the area of muscle or joint damage by, e.g., an intra-muscular or intra-articular injection. In one embodiment, pharmaceutical compositions comprising MPs of the invention may be introduced into the area of muscle or joint damage by an intramuscular or intra-articular injection to treat muscle or joint damage. In another embodiment, a pharmaceutical dosage form comprising MPs of the invention may be utilized to deliver MPs of the invention to the site of muscle or joint damage to treat muscle, tendon, ligament or joint damage.

In one aspect of the invention, the present application describes MPs comprising SDF-1α. In one embodiment, the application describes carbohydrate-based, e.g., GAG-based, e.g., heparin-based, degradable MPs comprising SDF-1α. In another aspect, the present application describes a method of recruiting pro-regenerative cells to, and/or reducing inflammation at, a muscle, joint, tendon, or ligament following injury, e.g., muscle injury such as rotator cuff injury, e.g., rotator cuff tear, by introducing MPs comprising an effective amount of SDF-1α to the site of muscle damage. In yet another aspect, the present application describes a method of treating a muscle, joint, tendon, or ligament injury, e.g., muscle injury such as rotator cuff injury, e.g., rotator cuff tear, by introducing MPs comprising an effective amount SDF-1α to the site of the muscle, joint, tendon, or ligament injury.

In another aspect of the invention, the present application describes MPs comprising TSG-6. In one embodiment, the application describes carbohydrate-based, e.g., GAG-based, e.g., heparin-based, degradable MPs comprising TSG-6. In another aspect, the present application describes a method of reducing inflammation of a muscle, joint, tendon, or ligament of a subject, e.g., a joint, e.g., a knee, hip, shoulder, ankle, wrist, finger, or toe joint, by introducing MPs comprising TSG-6. In yet another aspect, the present application describes a method of treating muscle, joint, tendon, or ligament damage in a subject in need of such treatment, e.g., joint damage caused by osteoarthritis (OA), by introducing MPs comprising TSG-6 to the site of muscle, joint, tendon, or ligament damage.

These and other aspects of the present invention will become apparent to those skilled in the art after a reading of the following detailed description of the invention, including the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C depict exemplary degradable 10 wt % N-desulfated heparin microparticle fabricated according to the invention. FIG. 1A shows microparticles fabricated with 10 wt % N-desulfated heparin methacrylamide, 90 wt % linear poly (ethylene glycol) diacrylate and 35 mM dithiothreitol (DTT). FIG. 1B is a phase image indicating that, in this non-limiting example, the depicted microparticles are ˜62±65 μm in diameter; black arrows indicate microparticles; scale bar is 100 FIG. 1C depicts SDF-1α release of ˜155 ng SDF-1α from 10 wt % Hep−^{-N} microparticles occurring over 1-3 days in vitro; n=3-5±SD.

FIGS. 2A-2B illustrate the experimental design: AlexaFluor633 tagged Hep−^{-N} is crosslinked within 10 wt % Hep−^{-N} microparticles and injected into the supraspinatus muscle immediately following injury, then the microparticles are tracked at day 3 and 7 via confocal microscopy. FIG. 2B shows that non-degradable microparticles (red) remain within the muscle (nuclei in blue) for at least 7 days (panels i and ii) while degradable microparticles appeared to have degraded by day 7 (panels iii and iv); the white arrow heads indicate microparticles; the scale bar is 100 μm.

FIGS. 3A-3B depict that SDF-1α remained near the supraspinatus muscle for at least 3 days following injury and injection. FIG. 3A shows representative images showing AF633 SDF-1α in the supraspinatus muscle over time (panels i through iv); black dotted circles indicate the region of interest used for quantification. FIG. 3B is a graph showing the AF633 SDF-1α signal as a percentage of the day 0 signal over 7 days and indicates that the highest AF633 SDF-1α signal was observed at day 1, and AF633 SDF-1α was still detected after at least 3 days; n=3±SD.

FIGS. 4A-4H illustrate that SDF-1α-loaded microparticles recruit significantly more M2-like macrophages 7 days following injury and treatment. FIGS. 4A-4D illustrate that no significant increase in cell recruitment was observed after 3 days except in total myeloid cells (FIG. 4A) and total macrophages (FIG. 4B) after treatment with unloaded microparticles. FIGS. 4E-4F illustrate that by day 7, significantly more total myeloid cells and macrophages are recruited to all experimental groups. No significant increase in recruitment of M1 macrophages is observed (FIG. 4G). FIG. 4H shows SDF-1α loaded microparticles recruited significantly more M2 macrophages than all other groups. The hash or pound symbol refers to significantly greater than contralateral control at that time point. The asterisk refers to significantly different; p⊏0.05; n=4-9±SD.

FIGS. 5A and 5B show that SDF-1α-loaded microparticles recruit significantly more mesenchymal stem cells 7 days following injury and treatment. FIG. 5A shows no significant increase in mesenchymal stem cell (MSC, CD29+CD44+CD90+) recruitment observed after 3 days. FIG. 5B shows that by day 7, significantly more MSCs are recruited to the SDF-1α-loaded microparticle group than all other groups. The hash or pound symbol refers to significantly greater than contralateral control; the asterisk refers to significantly different; p□0.05; n=4-11±SD.

FIGS. 6A-6C depict vascular looping observed in SDF-1α-loaded MP-treated supraspinatus muscle. FIGS. 6A and 6B show vascular staining present in the injured muscle compared to the uninjured control. FIG. 6C shows supraspinatus muscles treated with SDF-1α-loaded MPs exhibit CD31+ vascular looping 7 days following injury and treatment; blue=Hoechst stained nuclei, green=CD31+ vasculature; scale bar is 50 μm; n=2.

FIGS. 7A-7B show ¹H NMR of poly (ethylene glycol) diacrylate (PEGDA) and N-desulfated heparin methacrylamide (Hep^{-N}MAm). FIG. 7A shows characteristic peaks for acrylate groups present in the PEGDA spectra, and FIG. 7B shows characteristic peaks for methacrylamide peaks present in the N-desulfated heparin methacrylamide. Proton nuclear magnetic resonance (¹H NMR) was performed to determine the degree of PEGDA and Hep^{-N}MAm functionalization, whereby each material was dissolved at 10 mg/mL in deuterated H₂O (Sigma), run on a Bruker Avance III spectrometer at 400 Hz, and analyzed using iNMR software (Seto S P, Miller T, Temenoff J S. Effect of Selective Heparin Desulfation on Preservation of Bone Morphogenetic Protein-2 Bioactivity after Thermal Stress. Bioconjug Chem. 2015; 26(2):286-293).

FIG. 8 shows that 10 wt % Hep−^{-N} microparticles degrade between 8 to 30 days in vitro. A microparticle degradation time course in vitro, as described in Example 15, is dependent on the concentration of dithiothreitol (DTT) crosslinked within microparticles. Black arrows indicate microparticles; the scale bar is 100 μm.

FIGS. 9A-9C show the results of a study performed in Example 10 in which the supraspinatus muscle weight significantly decreases after 6 weeks and fibrosis significantly increased after 3 weeks following injury, compared to uninjured control. FIG. 9A shows that injured supraspinatus muscle weight is ˜40% the weight of the uninjured contralateral control 6 weeks following injury. The double asterisk refers to significantly different than uninjured control; p□0.05; n=5±SD. FIG. 9B shows Masson's trichrome staining used to identify fibrous infiltrate (blue) within the supraspinatus muscle (red); the scale bar is 100 μm. FIG. 9C shows image analysis indicating that fibrosis is significantly greater in the injured muscle compared to the contralateral control after 3 and 6 weeks. The double asterisk refers to significantly different than 3 and 6 week injury; p□0.05. The triple asterisk refers to significantly different than 1, 3, and 6 week injury; p□0.05; n=4-5±SD.

FIGS. 10A-10H depict flow cytometry gating scheme for inflammatory cell populations, as described in Example 11. FIG. 10A depicts cells first gated through forward scatter (FSC) area vs. side scatter (SSC) area plot. FIG. 10B depicts cells further gated through FSC-area vs. FSC-height plot to identify single cells. Fluorescence minus one (FMO)s are used to create gates for CD11b+ (FIG. 10C), CD68+ (FIG. 10D), and CD163+ cells (FIG. 10E). Example data from supraspinatus muscle shows the identification of leukocytes, CD11b+ (FIG. 10F), macrophages, CD11b+CD68+ (FIG. 10G), and M1 or M2 macrophages, CD11b+CD68+CD163−/+ (FIG. 10H).

FIGS. 11A-11C illustrate how degree of heparin sulfation affects TSG-6 bioactivity in vitro. FIGS. 11A-11B show that fully sulfated and N-desulfated heparin significantly enhances TSG-6 anti-plasmin activity, whereas FIG. 11C shows that fully desulfated heparin has no effect on TSG-6 activity compared to soluble TSG-6 controls. The asterisk refers to significantly different than all other groups; p≤0.05. The hash or pound symbol refers to not significantly different from each other but significantly different than all other groups; p≤0.05; n=3±SD.

FIGS. 12A-12B depict 10 wt % Hep−^{-N} MPs releasing TSG-6 over about 3 days and enhanced TSG-6 bioactivity. FIG. 12A shows that over 5 days, ˜1.5 μg TSG-6 is released from MPs; n=3-4±SD. FIG. 12B shows that TSG-6 released from MPs exhibits significantly more anti-plasmin activity than soluble TSG-6 at the same concentration. The asterisk refers to significantly lower than all other groups; p≤0.05; n=3-4±SD.

FIGS. 13A-13K show that TSG-6-loaded MPs reduce cartilage damage 3 weeks following MMT injury. FIGS. 13A-13D depict Safranin-O stained coronal sections of tibiae 3 weeks following injury and treatment, indicating that proteoglycan loss is observed in the injured (FIG. 13A) and soluble TSG-6 group (FIG. 13B), but not in the TSG-6 loaded MP group (FIG. 13C). FIGS. 13E-13H show contrast-enhanced μCT imaging of the same samples indicating that cartilage fibrillation is present in the (FIG. 13E) and soluble TSG-6 groups (FIG. 13F), but not present in the TSG-6 loaded MP group (FIG. 13G). Scale bars are 500 μm; n=3-4±SD. FIGS. 13I-13K shows quantified evaluation of articular cartilage indicating that cartilage thickness (FIG. 13I) and volume (FIG. 13J) are significantly increased compared to uninjured controls after injury and soluble TSG-6 treatment, and cartilage attenuation (FIG. 13K) is significantly increased after soluble TSG-6 treatment. In contrast, no significant increase in cartilage thickness, volume, or attenuation is observed in the TSG-6 loaded MP group compared to uninjured controls. The asterisk refers to significantly different than contralateral control; p≤0.05; n=3-4±SD.

FIGS. 14A-14B show small molecule (FTY720) loading and release from degradable heparin-based MPs. FIG. 14A shows the loading efficiency of the small molecule FTY720 varying between 35-80% of the originally added amount of FTY720. FIG. 14B shows the total amount of FTY720 released from MPs varying between about 0.5-5 μg over 7 days.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The phrases “parenteral administration”, “administered parenterally” and “administer parenterally” as used herein refer to modes of administration other than enteral and topical administration, usually by injection, and include, without limitation, intravenous (IV), intra-muscular, intra-articular, intraarterial, intrathecal, intracapsular, intraorbital, intracardiac, intradermal, intraperitoneal, transtracheal, subcutaneous, subcuticular, subcapsular, subarachnoid, intraspinal, epidural, and intrasternal injection and infusion. Hep^{-N}MAm refers to N-desulfated heparin methacrylamide, PEGDA refers to poly (ethylene glycol) diacrylate, DTT refers to dithiothreitol, MPs refers to microparticles, SDF-1a refers to stromal cell-derived factor-1alpha, TSG-6 refers to tumor necrosis factor-la stimulated gene-6, MMT refers to medial meniscal transection model, GAG refers to glycosaminoglycan, BSA refers to bovine serum albumin, PBS refers to phosphate buffer saline,

In one aspect, the present invention describes microparticles (MPs) comprising a protein and/or a small molecule. In one embodiment, the MPs of the invention may be carbohydrate-based, i.e., comprising a carbohydrate-based body. In one embodiment, the carbohydrate-based MPs of the invention may be GAG-based. In one embodiment, the MPs of the invention may be heparin-based MPs, i.e., N-desulfated heparin (Hep^{-N}) may be incorporated within the microparticles of the invention. Without wishing to be bound by theory, it is postulated that Hep^{-N} exhibits diminished anti-coagulant properties while maintaining the ability to bind protein and/or small molecule and protect its bioactivity. In another embodiment, dithiothreitol (DTT) may be incorporated within the MPs. Without wishing to be bound by theory, it is postulated that DTT may help vary the rate of hydrolytic degradation and allow for more complete release of protein and/or small molecule over time. In another embodiment, poly (ethylene glycol)-based linker, e.g. poly (ethylene glycol) diacrylate (PEGDA)-based linker may be utilized to incorporate DTT into heparin-based MPs. In one embodiment, the MPs of the invention may incorporate a protein active agent. In one embodiment, the protein incorporated within the MPs of the invention may comprise SDF-1α. In another embodiment, the protein incorporated within the MPs of the invention may comprise TSG-6. In another embodiment, the MPs of the invention may incorporate a small molecule active agent. In one embodiment, the small molecule active agent may be a hydrophobic small molecule, e.g., FTY720.

N-desulfated heparin (Hep^{-N}) can be prepared by dissolving a heparin sodium salt in water, passaging through a resin, and pH adjusting with pyridine as necessary. Water can be removed by evaporation. A heparin pyridinium salt solution can be frozen in liquid nitrogen, lyophilized, and then dissolved in DMSO, dialyzed and lyophilized. Hep^{-N} can be added to N-(3-Aminopropyl) methacrylamide hydrochloride to form Hep^{-N}MAm.

Microparticles can be formed via a water-and-oil emulsion of with Hep^{-N}MAm, PEGDA, DTT and a BSA/PBS solution in an aqueous phase. The DTT facilitates degradability. Proteins and/or small molecules can be loaded onto such microparticles by incubating the proteins and/or small molecules with the microparticles.

In another aspect, pharmaceutical compositions comprising MPs of the invention are described. In one embodiment, pharmaceutical compositions of the invention comprise heparin-based MPs comprising a protein and/or a small molecule and a pharmaceutically acceptable carrier, additive, or excipient.

In another aspect, pharmaceutical dosage forms comprising MPs of the invention are described. In various embodiments, pharmaceutical dosage forms of the invention may be liquid, semi-solid, powder, or solid dosage forms. In one embodiment, pharmaceutical dosage forms of the invention are liquid injectable dosage forms. The liquid injectable dosage forms may comprise one or more of an excipient, a carrier, a buffer, and a salt.

In another aspect, the present invention describes a method of recruiting pro-regenerative cells to the site of muscle, tendon, ligament or joint damage. In this aspect, the method of the invention comprises introducing MPs of the invention comprising an effective amount of a protein and/or a small molecule active agent into the area of muscle, tendon, ligament or joint damage by, e.g., an intramuscular or intra-articular injection. In one embodiment, pharmaceutical compositions comprising MPs of the invention comprising an effective amount of a protein and/or a small molecule active agent are introduced into the area of muscle, tendon, ligament or joint damage by injection, e.g. by an intramuscular or intra-articular injection, to recruit pro-regenerative cells to the site of muscle or joint damage. In another embodiment, a pharmaceutical dosage form comprising MPs of the invention may be utilized to deliver MPs of the invention to the site of muscle, tendon, ligament or joint damage to recruit pro-regenerative cells to the site of muscle, tendon, ligament or joint damage.

In another aspect, the present invention describes a method of treating muscle, tendon, ligament or joint damage by introducing MPs of the invention comprising an effective amount of a protein and/or a small molecule active agent into the area of muscle, tendon, ligament or joint damage by, e.g., an intra-muscular or intra-articular injection. In one embodiment, pharmaceutical compositions comprising MPs of the invention may be introduced into the area of muscle, tendon, ligament or joint damage by an intra-muscular or intra-articular injection to treat muscle, tendon, ligament or joint damage. In another embodiment, a pharmaceutical dosage form comprising MPs of the invention may be utilized to deliver MPs of the invention to the site of muscle, tendon, ligament or joint damage to treat muscle, tendon, ligament or joint damage.

In one aspect of the invention, the present application describes MPs comprising SDF-1α. In one embodiment, the application describes carbohydrate-based (e.g., GAG-based, e.g., heparin-based) degradable MPs comprising SDF-1α. In another aspect, the present application describes a method of recruiting pro-regenerative cells to muscle following rotator cuff injury, e.g., rotator cuff tear, by introducing MPs comprising an effective amount of SDF-1α to the site of muscle damage. In yet another aspect, the present application describes a method of treating a rotator cuff injury, e.g., rotator cuff tear, by introducing MPs comprising an effective amount SDF-1α to the site of the rotator cuff injury. The methods can be applied to treat any number of other muscle injuries, tendon injuries, ligament injuries, joint injuries, etc.

In another aspect of the invention, the present application describes MPs comprising TSG-6. In one embodiment, the application describes carbohydrate-based (e.g., GAG-based, e.g., heparin-based), degradable MPs comprising TSG-6. In another aspect, the present application describes a method of reducing inflammation at a joint of a subject, e.g., a knee, hip, shoulder, ankle, wrist, finger, or toe joint, by introducing MPs comprising TSG-6. In yet another aspect, the present application describes a method of treating joint damage in a subject in need of such treatment, e.g., damage caused by osteoarthritis (OA), by introducing MPs comprising TSG-6 to the site of joint damage. The method can be applied to treat any number of other joint injuries, tendon injuries, ligament injuries, etc.

In one embodiment, the microparticles of the invention may be between about 10 and about 500 micrometers in diameter, or between about 20 and about 100 micrometers in diameter, or between about 30 and about 80 micrometers in diameter.

Desulfated heparin derivatives that range from 0 to 100% total sulfation can differentially effect, and is some cases enhance, TSG-6 bioactivity in vitro. To facilitate delivery of TSG-6 into a joint, the TSG-6 may be carried by way of hydrolytically degradable heparin-based microparticles (MPs) that are injected into or near the joint via intra-articular injection. Heparin with sulfation of at least 10%, 15%, 20%, 25%, 30%, 35%, 40%, 45%, 50%, 55%, 60%, 65%, 70%, 75%, 80%, 85%, or 90% may be used. Without wishing to be bound by theory, heparin of greater total sulfation can enhance TSG-6 bioactivity. TSG-6 loaded on MPs with the appropriate sulfation level and delivered via intra-articular injection can reduce cartilage damage following MMT injury significantly more than soluble TSG-6 treatment.

Heparin is a naturally derived and highly sulfated glycosaminoglycan (GAG) that can bind to a myriad of positively charged proteins including TSG-6, as an injectable biomaterial carrier. The effect of heparin desulfation of heparin on TSG-6 binding and bioactivity has yet to be determined. Therefore, investigating the ability for desulfated heparin derivatives to maintain or enhance TSG-6 bioactivity is important to the development of an efficacious delivery strategy for TSG-6.

In another aspect, albumin may be used to entrap a hydrophobic small (e.g., less than 40, or less than 30, or less than 20 carbon atoms) molecule within MPs of the invention. Any hydrophobic small molecule may be used. In one embodiment, a sphingosine analog FTY720 may be incorporated into MPs of the invention.

### Co-Delivery Systems

In another aspect, bifunctional MPs have been designed to enhance the recruitment of endogenous pro-regenerative cells. To overcome the challenge of co-delivering two physiochemically distinct molecules (a large hydrophilic protein such as SDF-1α and a hydrophobic small molecule such as the sphingosine analog FTY720), a dual affinity MP has been engineered that exploits the growth factor affinity of a heparin derivative (Hep^{-N}) and lipid chaperone activity of albumin. FTY720 and SDF-1α may be successfully loaded and co-released from the Hep^{-N}-functionalized PEGDA MPs while maintaining bioactivity. Although in vivo delivery of FTY720 or SDF-1α individually promotes the enhanced recruitment of Ly-6Clow anti-inflammatory monocytes, co-delivery enhances the early accumulation and persistence of the differentiated wound healing CD206+ macrophages in the damaged tissue. Co-delivery may similarly promote the synergistic expansion of vasculature at the injection site, a key step in tissue healing.

In another aspect of the invention is provided pharmaceutical compositions comprising microparticles. The microparticles comprise protein and/or small molecule active agents optionally in combination with a pharmaceutically acceptable carrier, additive or excipient. One exemplary small molecule is the sphingosine analog FTY720. The pharmaceutical compositions comprising an effective amount of microparticles of the invention may be formulated as a pharmaceutical dosage form for administration to a subject.

### Pharmaceutical Compositions and Dosage Forms

Also provided are pharmaceutical compositions comprising the microparticles described herein. When employed as pharmaceuticals, the microparticles can be administered in the form of pharmaceutical compositions which is a combination of the microparticles of the invention and a pharmaceutically acceptable carrier. These compositions can be prepared in a manner well known in the pharmaceutical art, and can be administered by a variety of routes. Such pharmaceutical compositions can be administered locally or systemically. The term “systemic” as used herein includes parenteral, topical, transdermal, oral, by inhalation/pulmonary, rectal, nasal, buccal, and sublingual administration. The term “parenteral” as used herein includes subcutaneous, intradermal, intravenous, intramuscular, intracranial, and intraperitoneal administration. The compounds may be administered intramuscularly, intra-articularly, or subcutaneously at the damage site in therapeutically effective amounts to treat muscle, tendon, ligament or joint damage.

Pharmaceutical compositions containing microparticles of the invention can be prepared in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, injectable solutions, including sterile injectable solutions, and sterile packaged powders.

In some embodiments, the pharmaceutical composition of the invention is in liquid form. Liquid forms include, by way of non-limiting example, emulsions, solutions, suspensions, syrups, slurries, dispersions, colloids and the like. In some embodiments, a pharmaceutical composition described herein is in liquid, semi-solid or solid (e.g., powder) form. In specific embodiments, a pharmaceutical composition described herein is in semi-solid form, e.g., a gel, a gel matrix, a cream, a paste, or the like. In some embodiments, semi-solid forms comprise a liquid vehicle.

In some embodiments, pharmaceutical compositions of the invention can include one or more pharmaceutically acceptable carriers. As used herein the term “pharmaceutically acceptable carrier” is intended to include any and all solvents, excipients, dispersion media, coatings, antibacterial and antifungal agents, isotonic and adsorption delaying agents, and the like, compatible with pharmaceutical administration. Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X). Except insofar as any conventional media or agent is incompatible with the active compound, such media can be used in the compositions of the invention. Supplementary active compounds can also be incorporated into the compositions.

Sterile injectable solutions can be prepared by incorporating microparticles of the invention in an appropriate solvent with one or a combination of ingredients enumerated above, as required, followed by filter sterilization. Generally, dispersions are prepared by incorporating the microparticles into a sterile vehicle which contains a basic dispersion medium and the required other ingredients from those enumerated above. In the case of sterile powders for the composition of sterile injectable solutions, the preferred methods of composition are vacuum drying and freeze-drying, e.g., lyophilization, which yields a powder plus any additional desired ingredient from a previously sterile-filtered solution thereof.

The microparticles can be prepared with materials that will protect them against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The microparticles of the invention comprise active proteins and/or small molecule compounds and can be effective over a wide dosage range. They are generally administered in a pharmaceutically effective amount. It will be understood, however, that the amount of the microparticles actually administered will usually be determined by a physician, according to the relevant circumstances, including the condition to be treated, the chosen route of administration, the actual microparticles administered, the age, weight, and response of the individual patient, the severity of the patient's symptoms, and the like.

In some embodiments, a composition or unit dosage form described herein is administered as an emulsion, a solution, a suspension, a syrup, a slurry, a dispersion, a colloid, a a capsule, a gel capsule, a semi-solid, a solid forma gel, a gel matrix, a cream, a paste, a tablet, a granule, a sachet, a powder, or the like. In certain aspects, about 0.0001 mg to about 2000 mg, about 0.01 mg to about 1000 mg, or about 0.1 mg to about 750 mg, about 1 mg to about 500 mg, about 10 mg to about 250 mg, about 20 mg to about 150 mg of microparticles per day or per dose is administered to an individual.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the microparticle preparations typically will be between 3 and 11, or from 5 to 9, or from 6 to 8.5.

The therapeutic dosage of the microparticles of the invention can vary according to, for example, the particular type and location of the injury for which the treatment is made, the manner of administration of the microparticles, the health and condition of the patient, and the judgment of the prescribing physician. The proportion or concentration of the microparticles of the invention in a pharmaceutical composition can vary depending upon a number of factors including dosage, chemical characteristics (e.g., hydrophobicity), and the route of administration. The dosage is likely to depend on such variables as the type and severity of the injury, the overall health status of the particular patient, the relative biological efficacy of the compound selected, formulation of the excipient, and its route of administration. Effective doses can be extrapolated from dose-response curves derived from in vitro or animal model test systems.

The present application also includes pharmaceutical kits useful, for example, in the treatment of muscular and/or articular damage. The pharmaceutical kits may include one or more containers containing a pharmaceutical composition comprising a therapeutically effective amount of the microparticles of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

Delivery devices are important not only for delivering the microparticles of the invention, but also for providing an appropriate environment for storage. This would include protection from microbial contamination and chemical degradation. The device and formulation should be compatible so as to avoid potential leaching or adsorption. The delivery device (or its packaging) can be optionally provided with a label and/or with instructions for use indicating that the composition should be used, e.g., intramuscularly or intra-articularly.

### Packaging and Delivery

The microparticles described herein can be administered with various medical devices. For example, microparticles described herein can be administered with a needle-less hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules also are known.

The compositions comprising microparticles described herein can be packaged in a two chamber syringe. For example, the microparticles in lyophilized form can be placed into a first syringe chamber and a liquid can be present in a second syringe chamber (see e.g., U.S. Published Application No. 2004-0249339).

The microparticle compositions described herein can be packaged in a needleless syringe (see e.g., U.S. Pat. Nos. 6,406,455 and 6,939,324). Briefly, as one example, the injection device includes: a gas chamber containing a gas or a source of gas; a port which can allow for release of gas from the gas chamber; a plunger, which upon the release of gas from the gas chamber, can cause movement of at least a first piston; a first piston; a second piston; a first chamber, e.g. a chamber useful for drug storage and mixing; a piston housing, in which are disposed the first piston, the second piston and the first chamber; a displacement member which can, independent of the motive power of gas from the gas chamber, cause movement of one or both of the first and second pistons (the displacement member can be the plunger or a separate member); an orifice suitable for needleless injection in communication with the first chamber; wherein the first and second piston, are slidably disposed within the piston housing, and the displacement member, the source of gas, and the plunger are disposed such that: in a first position of the pistons, a second chamber, e.g., a fluid reservoir, is defined within the piston housing by the first piston, the piston housing and the second piston, the displacement member can move one or both of the pistons into a second position wherein the first piston is in a position such that the second chamber, which can be a fluid reservoir, is in communication with the first chamber, which can be a drug storage and mixing chamber, and the second piston is moved in the direction of the first piston, thereby decreasing the volume of the second chamber and allowing the transfer of fluid from the second chamber to the first chamber, the plunger, upon release of gas from the gas chamber, causes the first piston to move so as to decrease the volume of the first chamber allowing a substance to be expelled through the orifice and from the chamber and, e.g., to a subject.

The needleless syringe can include separate modules for a first component, e.g., a dry or liquid component, and a second component, e.g., a liquid component. The modules can be provided as two separate components and assembled, e.g., by the subject who will administer the component to himself or herself, or by another person, e.g., by an individual who provides or delivers health care. Together, the modules can form all or part of the piston housing of devices described herein. The devices can be used to provide any first and second component where it is desirable to store or provide the components separately and combine them prior to administration to a subject.

### Methods of Use

For administration into human patients, protein-and/or small molecule-loaded microparticles would be delivered via intra-muscular injection using standard needles and syringes through the skin of the patient into the muscle of interest. This approach is applicable to most muscles accessible via injection through the skin, and could be applicable to other animal species including dogs, horses, and other animals using the same procedure. For administration into joint spaces, protein-and/or small molecule-loaded microparticles would be loaded into standard needles and syringes. Next, the protein-loaded microparticles would be injected through the skin into the joint space, and a similar procedure could be used for most joint spaces. For administration into tendons, protein-loaded microparticles would be loaded into standard needles and syringes. Next, the protein-loaded microparticles would be injected through the skin into the tendon, and a similar procedure could be used for most tendons accessible through the skin. For administration into ligaments, protein-loaded microparticles would be loaded into standard needles and syringes. Next, the protein-loaded microparticles would be injected through the skin into the ligament, and a similar procedure could be used for most ligaments accessible through the skin. Many tendons and ligaments are too deep for a skin injection. In the case a tendon or a ligament cannot be injected through the skin, an injection may be performed as part of a surgical procedure to expose the injured tissue. Furthermore, though the exact location of injection may vary based on the animal, this injection procedure can be utilized for other animal species including dogs, horses, and other animals.

### ADDITIONAL EMBODIMENTS

1. A degradable microparticle comprising a GAG-based (e.g., heparin-based) body and an active agent selected from a protein and a small molecule, wherein the active agent is positively charged.
2. The degradable microparticle of embodiment 1, wherein the GAG-based body is an N-desulfated heparin-based body.
3. The degradable microparticle of embodiments 1 or 2, wherein the degradable microparticle further comprises DTT.
4. The degradable microparticle of any of embodiments 1-3, wherein the degradable microparticle further comprises a poly (ethylene glycol)-based linker.
5. The degradable microparticle of any of embodiments 1-4, wherein the poly (ethylene glycol)-based linker is a PEGDA-based linker.
6. The degradable microparticle of any of embodiments 1-5, wherein the active agent is a protein or a small molecule, e.g., FTY720.
7. The degradable microparticle of any of embodiments 1-6, wherein the active agent is SDF-1α.
8. The degradable microparticle of any of embodiments 1-6, wherein the active agent is TSG-6.
9. The degradable microparticle of any of embodiments 1-8, wherein the degradable microparticle is between about 10 and about 500 micrometers in diameter.
10. The degradable microparticle of any of embodiments 1-8, wherein the degradable microparticle is between about 20 and about 100 micrometers in diameter.
11. The degradable microparticle of any of embodiments 1-8, wherein the degradable microparticle is between about 30 and about 80 micrometers in diameter.
12. The degradable microparticle of claim 11, wherein the GAG-based body is an N-desulfated heparin-based body, the degradable microparticle further comprises DTT and a PEGDA-based linker, the active agent is selected from SDF-1α, TSG-6, FTY720 and combinations thereof.
13. A pharmaceutical composition comprising a plurality of the degradable microparticles of any of embodiments 1-12 and a pharmaceutically acceptable carrier, additive, or excipient.
14. A pharmaceutical dosage form comprising the degradable microparticle of any of embodiments 1-11 or the pharmaceutical composition of embodiment 13.
15. The pharmaceutical dosage form of embodiment 14, wherein the dosage form is a liquid injectable dosage form.
16. A method of fabricating a degradable microparticle, said method comprising forming a water-and-oil emulsion.
17. A method of fabricating a degradable Hep^{-N} microparticle comprising adding PEGDA and dithiothreitol (DTT) to a solution comprising bovine serum albumin (BSA) in PBS, incubating at 30-40° C. for 20-40 minutes, adding Hep^{-N}MAm to the solution, and incubating the aqueous phase for 20-40 minutes.
18. A method of recruiting pro-regenerative cells to, and/or reducing inflammation at, a site of muscle, tendon, ligament or joint damage, the method comprising introducing into the site of muscle or joint damage a degradable microparticle comprising a GAG-based (e.g., heparin-based) body and an amount of an active agent selected from a protein and a small molecule effective to
   (i) recruit pro-regenerative cells to the site of muscle, tendon, ligament or joint damage, and/or
   (ii) reduce inflammation at the site of muscle, tendon, ligament or joint damage.
19. The method of embodiment 18, wherein the degradable microparticle is introduced into the site of muscle or joint damage by an intramuscular or intra-articular injection.
20. The method of embodiments 18 or 19, wherein a pharmaceutical composition comprising a plurality of the degradable microparticles and a pharmaceutically acceptable carrier, additive, or excipient is introduced into the site of muscle or joint damage.
21. The method of any of embodiments 18-20, wherein the active agent is a protein.
22. The method of any of embodiments 18-21, wherein the damage is muscle damage.
23. The method of embodiment 22, wherein the muscle damage is rotator cuff damage and optionally wherein the active agent is SDF-1α.
24. The method of any of embodiments 18-21, wherein the damage is joint damage.
25. The method of embodiment 24, wherein the active agent is TSG-6.
26. The method of any of embodiments 18-25, wherein the degradable microparticle is between about 10 and about 500 micrometers in diameter.
27. The method of any of embodiments 18-26, wherein the degradable microparticle is between about 20 and about 100 micrometers in diameter.
28. The method of any of embodiments 18-27, wherein the degradable microparticle is between about 30 and about 80 micrometers in diameter.
29. A method of treating muscle or joint damage in a subject in need of such treatment, the method comprising introducing into the site of muscle or joint damage a degradable microparticle comprising a GAG-based (e.g., heparin-based) body and an amount of an active agent selected from a protein and a small molecule effective to treat muscle or joint damage.
30. The method of embodiment 29, wherein the degradable microparticle is introduced into the site of muscle or joint damage by an intramuscular or intra-articular injection.
31. The method of embodiments 29 or 30, wherein a pharmaceutical composition comprising a plurality of the degradable microparticles and a pharmaceutically acceptable carrier, additive, or excipient is introduced into the site of muscle or joint damage.
32. The method of any of embodiments 29-31, wherein the active agent is a protein.
33. The method of any of embodiments 31-32, wherein the damage is muscle damage.
34. The method of embodiment 33, wherein the muscle damage is rotator cuff damage and the active agent is SDF-1α.
35. The method of any of embodiments 29-32, wherein the damage is joint damage.
36. The method of embodiment 35, wherein the active agent is TSG-6.
37. The method of any of embodiments 29-32, 35, or 36, wherein the subject is suffering from osteoarthritis.
38. The method of any of embodiments 29-37, wherein the degradable microparticle is between about 10 and about 500 micrometers in diameter.
39. The method of any of embodiments 29-38, wherein the degradable microparticle is between about 20 and about 100 micrometers in diameter.
40. The method of any of embodiments 29-39, wherein the degradable microparticle is between about 30 and about 80 micrometers in diameter.
41. A method of reducing inflammation at a site of muscle, joint, tendon, or ligament damage, the method comprising introducing into the site of muscle, joint, tendon, or ligament damage a degradable microparticle comprising a GAG-based body and an amount of an active agent selected from a protein and a small molecule effective to reduce inflammation at the site of muscle, joint, tendon, or ligament damage.
42. The method of embodiment 41, wherein the joint damage is osteoarthritis and the active agent is TSG-6.
43. The method of embodiment 41 or 42, wherein the degradable microparticle is between about 30 and about 80 micrometers in diameter.

### EXAMPLES

The following examples illustrate specific aspects of the instant description. The examples should not be construed as limiting, as the examples merely provide specific understanding and practice of the embodiments and their various aspects.

### Example 1

### Heparin Modification

N-desulfated heparin (Hep^{-N}) was prepared as described previously. Tellier L E, Miller T, McDevitt T C, Temenoff J S. J Mater Chem B. 2015; Seto S P, Miller T, Temenoff J S. Bioconjug Chem. 2015; 26(2):286-293; Inoue Y, Nagasawa K. Carbohydr Res. 1976; 46:87-95; Nagasawa K, Inoue Y, Kamata T. Carbohydr Res. 1977; 58:47-55. Briefly, heparin sodium salt (Hep) from porcine intestinal mucosa (Sigma) was dissolved at 10 mg/mL in dH₂O and passed through Dowex 50WX4 resin (mesh size 100-200, Sigma). Pyridine was added drop-wise to the heparin until the solution reached pH 6 and the solution was placed on a rotatory evaporator (Buchi) to remove excess dH₂O and pyridine. Heparin pyridinium salt solution was frozen in liquid nitrogen, lyophilized, and then dissolved at 1 mg/mL in 9:1 v/v dimethyl sulfoxide (DMSO)/dH₂O at 50° C. for 2 hours. Following the reaction, the Hep^{-N} was cooled on ice and precipitated with 95% ethanol saturated with sodium acetate, then collected by centrifugation. The resulting material was dissolved in dH₂O, dialyzed for 3 days, lyophilized, frozen in liquid nitrogen, and stored at −20° C.

For methacrylamide (MAm) functionalization, 1.1 mM Hep^{-N}, 83.0 mM N-hydroxysulfosuccinimide (sulfo-NHS, Sigma), 101.0 mM N-(3-Aminopropyl) methacrylamide hydrochloride (APMAm, Polysciences Inc.), and 156.0 mM (N-3-Dimethylaminopropyl)-N′-ethylcarbodiimide hydrochloride (EDC, Sigma) were dissolved in 10 mL phosphate buffer saline (PBS, Teknova) solution. After stirring on ice for 6 hours, the Hep^{-N}MAm was dialyzed for 2 days, lyophilized, frozen in liquid nitrogen, and stored at −20° C.

To fluorescently label Hep^{-N}MAm, Hep^{-N}MAm was dissolved at 10 mg/mL in 0.1 M Na₂HPO₄ solution at pH 6. Next, 10 mM EDC and 5.7 μM AlexaFluor633 hydrazide (AF633, Invitrogen) were added and the reaction proceeded in the dark for 90 minutes at RT. AF633 Hep^{-N}MAm was dialyzed for 2 days, frozen in liquid nitrogen, lyophilized, and stored at −20° C.

### Example 2

### Poly(ethylene glycol)diacrylate Synthesis

Poly (ethylene glycol) (PEG, 3.4 kDa, Sigma) was reacted with acryloyl chloride (AcCl, Sigma) in an 8:1 AcCl to PEG molar ratio in dichloromethane (DCM) solution, as described in Van De Wetering P, Metters A T, Schoenmakers R G, Hubbell J A. J Control Release. 2005; 102(3):619-627. Triethylamine (TEA, Sigma) was added drop-wise in a 1:1 TEA to AcCl molar ratio as a catalyst to yield linear PEG-diacrylate (PEGDA).

### Example 3

### Proton Nuclear Magnetic Resonance

Proton nuclear magnetic resonance (¹H NMR) was performed to determine the degree of PEGDA and Hep^{-N}MAm functionalization, whereby each material was dissolved at 10 mg/mL in deuterated H₂O (Sigma), run on a Bruker Avance III spectrometer at 400 Hz, and analyzed using iNMR software. Seto S P, Miller T, Temenoff J S. Bioconjug Chem. 2015; 26(2):286-293.

### Example 4

### Strong Anion Exchange High Performance Liquid Chromatography

Strong anion exchange high performance liquid chromatography (SAX-HPLC) was performed at the University of Georgia Complex Carbohydrate Research Center (CCRC) to determine the disaccharide composition of Hep and Hep^{-N}. Hep and Hep^{-N} were dissolved at 12.5 mg/mL in a heparinase mixture of 0.5 U/mL heparinases I, II, and III for 24 hours at 37° C. The reaction was then quenched by heating the mixture for 2 minutes at 100° C.

SAX-HPLC was carried out on an Agilent system using a Waters Spherisorb analytical column (4.6×250 mm; 5 μm particle size) at 25° C. Analytes were detected by their UV absorbance at 232 nm using a buffer system consisting of 2.5 mM sodium phosphate (Na₃PO₄) and pH 3.5, which was gradually transitioned from 0 to 1.2 M NaCl. The flow rate was 1.0 mL/min and detection was performed by post-column derivatization and fluorescence detection. Commercial standard disaccharides (Dextra Laboratories) were used for identification of each disaccharide based on elution time and calibration.

### Example 5

### Size Exclusion Chromatography

Size exclusion high performance liquid chromatography (SEC-HPLC) was performed at the University of Georgia CCRC to analyze the average molecular weight and desulfation characteristics of Hep and Hep^{-N}. Hep and Hep were dissolved at 20 mg/mL in a 0.5 M lithium nitrate buffer. Separations were carried out using two TSKGel G2000SWXL columns (7.8 mm ID×30 cm), connected in series, on an Agilent 1200 LC instrument using refractive index detection and a sample flow rate and injection volume of 0.6 mL/min and 10 μL, respectively. Cirrus GPC software was used to construct 3rd order polynomial standard curves with the molecular weights and elution times of USP enoxaparin sodium molecular weight calibrants and broad standard USP heparin molecular weight calibrants. Weight molecular weights were calculated in Cirrus using the raw chromatograms exported from the SEC-HPLC instrument software.

### Example 6

### Microparticle Fabrication

10 wt % Hep microparticles (MPs) were fabricated via water-and-oil emulsion. First, 50.0 mg PEGDA and 0.92-1.85 mg dithiothreitol (DTT, 20-40 mM, Sigma) were added to 273 μL 10 wt % bovine serum albumin (BSA, Thermo) PBS solution and incubated at 37° C. for 30 minutes to allow for Michael Type addition between PEGDA and DTT. Next, 5.6 mg Hep^{-N}MAm was added and the aqueous phase was incubated for an additional 30 minutes. For fluorescently tagged Hep MPs (AF633 Hep^{-N} MPs), a 1:1 ratio of AF633 Hep^{-N}MAm to Hep^{-N}MAm was used and for non-degradable MPs, no DTT was added during fabrication.

After 27 μL of 0.05 wt % Irgacure 2959 photo initiator (Ciba) was added, the aqueous phase described above was added drop-wise to an oil phase of 5 mL mineral oil (Amresco) with 3.0-3.2 μL Span80 (TCI) and allowed to homogenize at 4000 RPM (Polytron PT 3100, Kinematica) for 5 minutes. The water-and-oil emulsion was nitrogen purged for 1 min then placed into a petri dish under UV (˜15 mW/cm²) for 10 minutes to allow for free radical polymerization between PEGDA and Hep^{-N}MAm. Finally, the MP solution was added to 35 mL dH₂O, centrifuged at 4000 RPM for 5 minutes, and the oil phase was removed. MPs were washed once more with dH₂O, then pipetted through 40 μm cell strainers to remove MPs under 40 μm in diameter.

Once fabricated, MPs were sterilized in 70% ethanol on rotary at 4° C. for 30 minutes, followed by three 30-minute washes in sterile PBS. MPs were imaged via phase microscopy and size distribution was measured using ImageJ software. MPs were stored in sterile PBS at 4° C. and used within 2 weeks of fabrication.

### Example 7

### SDF-1α Loading and Release from Microparticles

To load SDF-1α onto microparticles, 1.0-1.2 μg human SDF-1α (R&D Systems) was added to 0.6 mg MPs in 50 μL 0.1 wt % BSA solution. SDF-1α and MPs were incubated for 2 hours at 4° C., after which time MPs were rinsed by adding an additional 450 μL 0.1 wt % BSA solution. The MPs were centrifuged for 3 minutes at 10,000 RCF and 495 μL supernatant was removed.

For in vitro SDF-1α release studies, the removed supernatant was replaced with 495 μL fresh 0.1 wt % BSA solution and samples were incubated at 37° C. MPs were centrifuged and 495 μL supernatant was removed and replaced 3 hours, 1, 3, 7, 10 and 15 days following SDF-1α loading. SDF-1α protein levels were quantified with a human SDF-1α ELISA kit (R&D Systems) using the manufacturer's protocol, except for standard curves which were made with recombinant human SDF-1α rather than the provided standard. To ensure equivalent cumulative SDF-1α release for each in vivo study, an in vitro SDF-1α release study from MPs was conducted prior to each individual surgery; n=3-5 per release study.

### Example 8

### Rotator Cuff Injury Model

Rotator cuff injury was induced using a similar method to previously established protocols [39]. Male Sprague-Dawley rats (250-300 g initial weight and 8-10 weeks old) were used in accordance with protocols approved by the Georgia Institute of Technology Institutional Animal Care and Use Committee. Prior to surgery animals were anesthetized by 5% isoflurane (Isothesia), followed by 2-3% isoflurane during surgery and were administered sustained release buprenorphine as an analgesic. The left chest and arm were shaved, wiped with alcohol/chlorhexidine, and a ˜2 cm incision was made through the skin and deltoid, parallel to and just below the clavicle. To induce injury, a ˜5 mm portion of the suprascapular nerve was resected and, after orienting the humerus to expose the supraspinatus and infraspinatus tendon insertions, both tendons were sharply transected. The deltoid and skin were closed using Vicryl 4-0 absorbable sutures (Ethicon) and wound clips, respectively. The right rotator cuff of each animal served as an internal uninjured contralateral control.

### Example 9

### In Vivo Fluorescently-Tagged Microparticle Injection and Imaging

Non-degradable (0 mM DTT) and degradable (35 mM DTT) AF633 Hep^{-N} MPs were suspended in 120 μL sterile dH₂O (4.3 mg MPs) and subsequently loaded into sterile syringes with 20-gauge 1.5 in. hypodermic needles (BD Precision Glide). Immediately following tendon transection and denervation, the MPs were injected into the supraspinatus muscle located posterior to the scapula. Uninjured contralateral supraspinatus muscles were not injected with MPs and served as negative controls. After 3 and 7 days, supraspinatus muscles were dissected from the scapula, sliced in half length-wise, stained with a 1:1000 dilution Hoechst cellular stain (Thermo) in PBS for 5 minutes, and remained unfixed for imaging. Muscles were whole-mounted and single fluorescent images were obtained using a Zeiss LSM 700 confocal microscope with a 20× objective to visualize AF633 Hep^{-N} MPs (red) within the muscle tissue (blue); n=2 animals per group per time point.

### Example 10

### Histology and Quantification

After 1, 3, and 6 weeks following injury and treatment, animals were euthanized and the supraspinatus muscles were dissected, weighed, and fixed in 2% paraformaldehyde solution (PFA, J. T. Baker) for 45 minutes, followed by incubations in 10% v/v optimum cutting temperature (OCT, Sakura Finetek) PBS solutions with 0, 10, and 20% sucrose for 10 minutes each. Muscles were placed in histology blocks with 100% OCT under vacuum overnight and then frozen in 190 proof ethanol cooled by liquid nitrogen. Muscles were sectioned with a cryostat (Thermo Scientific CryoStar NX70) into 7μm sections perpendicular to the muscle to create tissue cross-sections. Masson's trichrome (Sigma, Fisher) was used to identify fibrous infiltrate and following staining, slides were mounted with Cytoseal 60 (Richard Allen Scientific) and cover slipped. Sections were imaged with a Nikon Eclipse 80i and analyzed using ImageJ software and color thresholding; n=4-5 per experimental group per time point. To quantify fibrous infiltration:

$$\% of fibrous infiltration = \frac{blue pixel count}{red pixel count + blue pixel count} \times 100$$

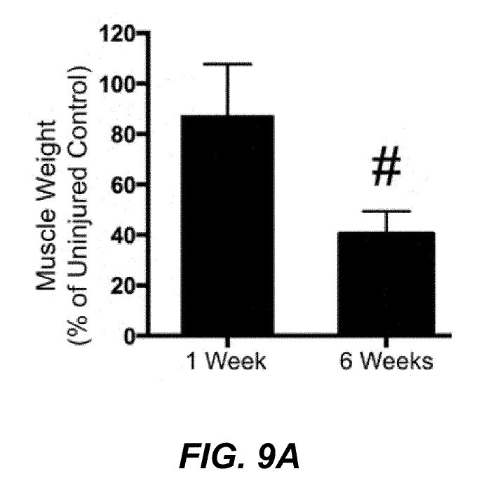

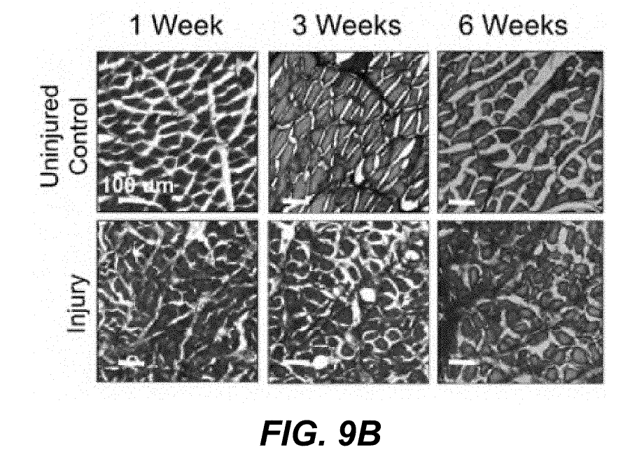

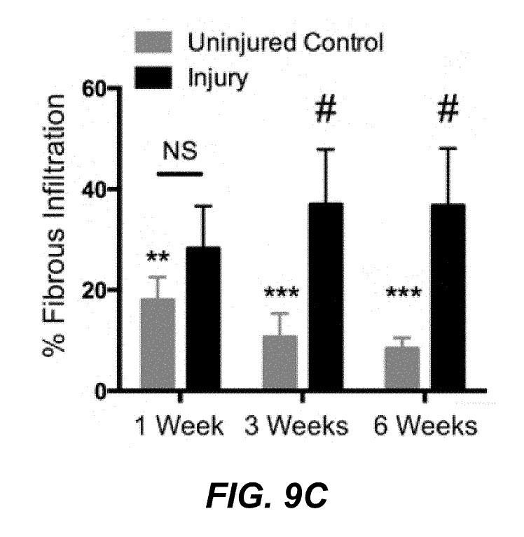

The data are shown in FIGS. 9(A-C), which together show that supraspinatus muscle weight significantly decreases after 6 weeks and fibrosis significantly increases after 3 weeks following injury, compared to uninjured control.

### Example 11

### In Vivo SDF-1α Loaded Microparticle Injection and Flow Cytometry

For flow cytometry experiments, groups included SDF-1α loaded MPs (0.6 mg MPs which released ˜155 ng SDF-1α in vitro), unloaded MPs (4.3 mg MPs), and injury only (no SDF-1α or MPs). For SDF-1α loaded MPs, MPs were loaded with SDF-1α as described above but after centrifugation, MPs were resuspended in a total volume of 120 μL sterile 0.1 wt % BSA solution. For unloaded MP controls, MPs were also suspended in a total volume of 120 μL sterile 0.1 wt % BSA solution but the maximum concentration of MPs (36 mg/mL) was used. MPs were then loaded into sterile syringes with 20-gauge 1.5 in. hypodermic needles and injected into the supraspinatus muscle immediately following tendon transection and denervation.

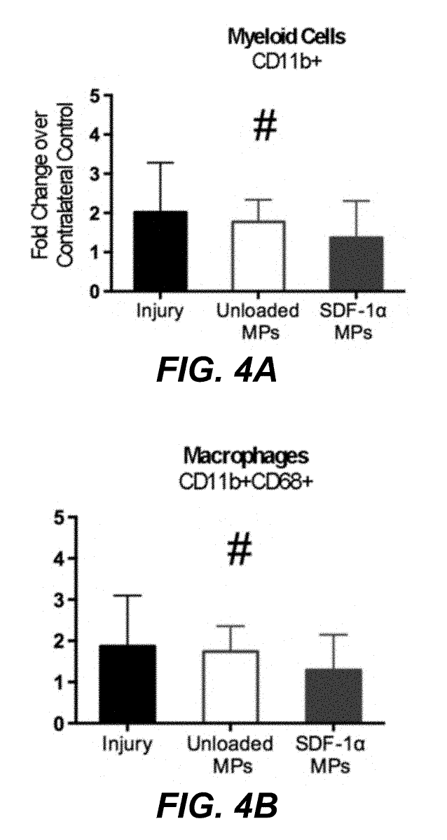

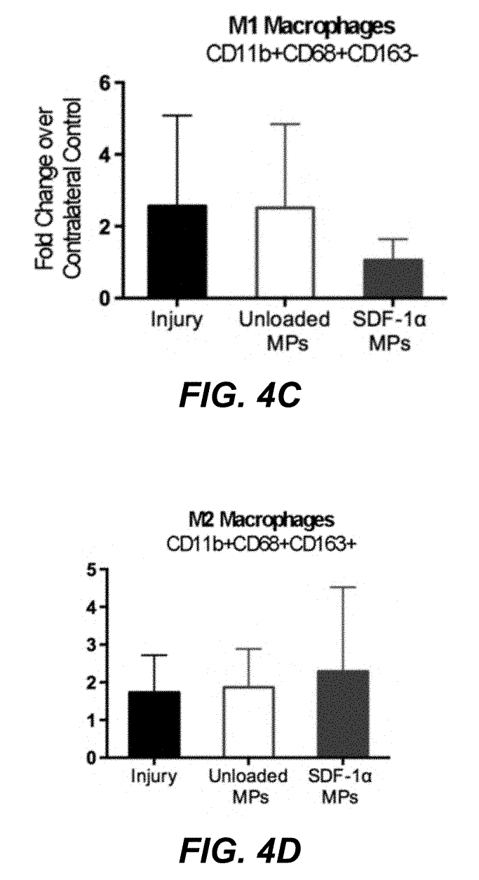

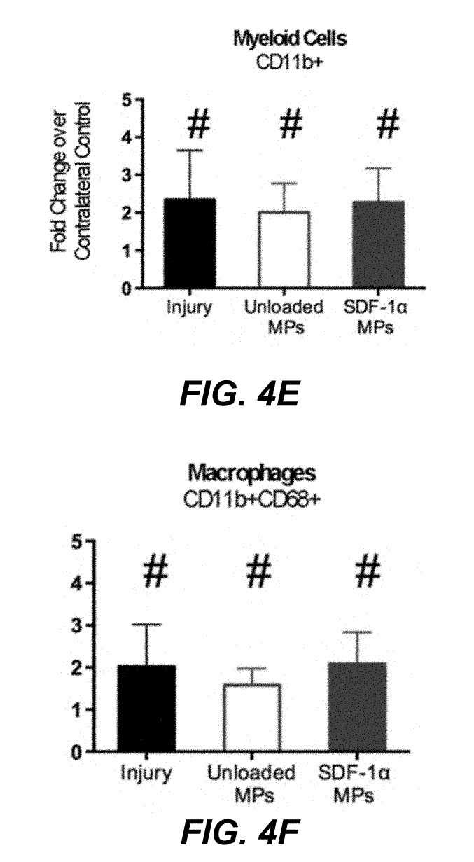

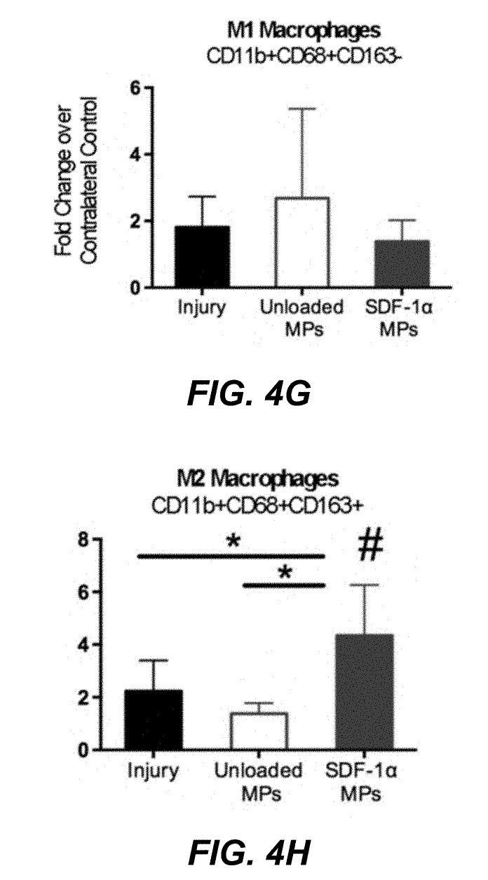

Supraspinatus muscles were harvested 3 and 7 days following injury and treatment, digested with collagenase 1A (Sigma) for 45 minutes at 37° C., and passed through a 40 μm cell strainer (Corning). One-half of each sample was stained with the inflammatory cell panel that included FITC-conjugated anti-CD11b (AbD Serotec), PE-conjugated anti-CD163 (BioRad), and APC-conjugated anti-CD68 (BioRad) and the other one-half was stained with the MSC panel that included PE-conjugated anti-CD29 (BioLegend), APC-conjugated anti-CD44 (BioLegend), and BV421-conjugated anti-CD90 (BioLegend). Samples were stained for 30 minutes with the appropriate antibodies and fixed in 2% PFA for 20 minutes, then analyzed using a FACS-AriaIIIu flow cytometer (BD Biosciences). For unloaded MPs, there was small but significant elevation in total leukocytes (1.8±0.6× compared to contralateral control) and macrophages (1.7±0.6×) compared to the uninjured contralateral controls after 3 days (FIGS. 4A-4B). For all other groups, there were no significant differences in leukocytes (1.3-2.1×), macrophages (1.2-1.9×), or macrophage subpopulations (1.1-2.6×) between the experimental groups and the uninjured contralateral controls after 3 days (FIGS. 4A-4D). There were also no differences between any of the experimental groups, including SDF-1α loaded MPs, unloaded MPs, and injury only at the day 3 time point.

In contrast, at 7 days following injury, significantly more M2-like macrophages were observed in muscle treated with SDF-1α loaded MPs than uninjured controls (4.3±1.9×), unloaded MPs (1.4±0.4×) and injury alone (2.2±1.2×, FIG. 4H). There were also no significant differences in M1-like macrophages between groups (FIG. 4G). Notably, significantly more total leukocytes (2.0-2.3×) and macrophages (1.6-2.1×) were detected in each experimental group as compared to the respective uninjured contralateral controls (FIGS. 4E-4F).

Inflammatory cells were identified as CD11b+ leukocytes, CD11b+CD68+ macrophages, and CD163 was used to differentiate M2-like (CD11b+CD68+CD163+) from M1-like macrophages (CD11b+CD68+CD163−) [7,40]. MSCs were identified as triple positive for CD29, CD44, and CD90 [41]; n=4-9 animals per group per time point. For data analysis, each cell population was first calculated as a percentage of single cells:

$$\% of single cells = \frac{\# of cells in subpopulation}{\# of single cells}$$

Then, each % of single cell value was divided by the % of single cells in the uninjured contralateral control of the same animal:

$$Fold \text{-} change over contralateral control = \frac{\% of single cells in experimental group}{\% of single cells in contralateral control}$$

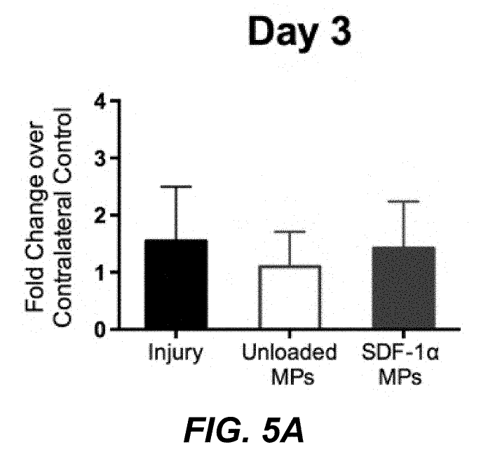

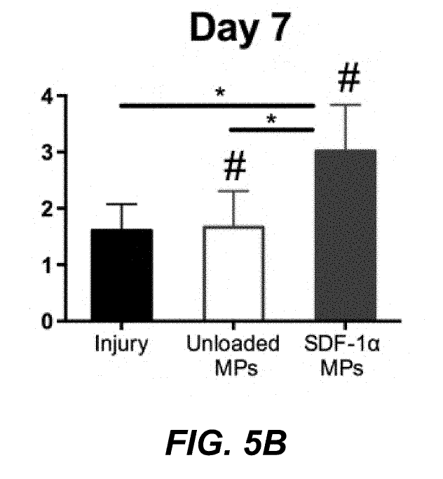

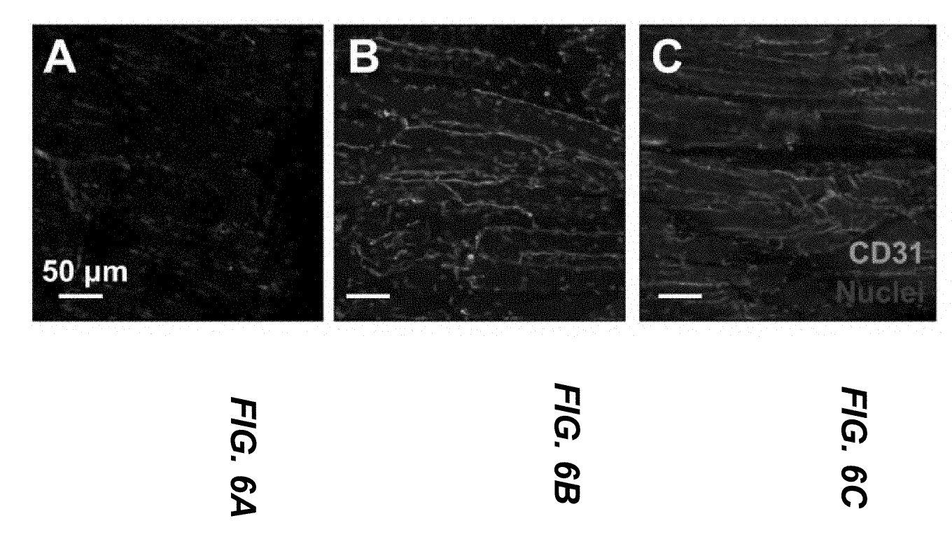

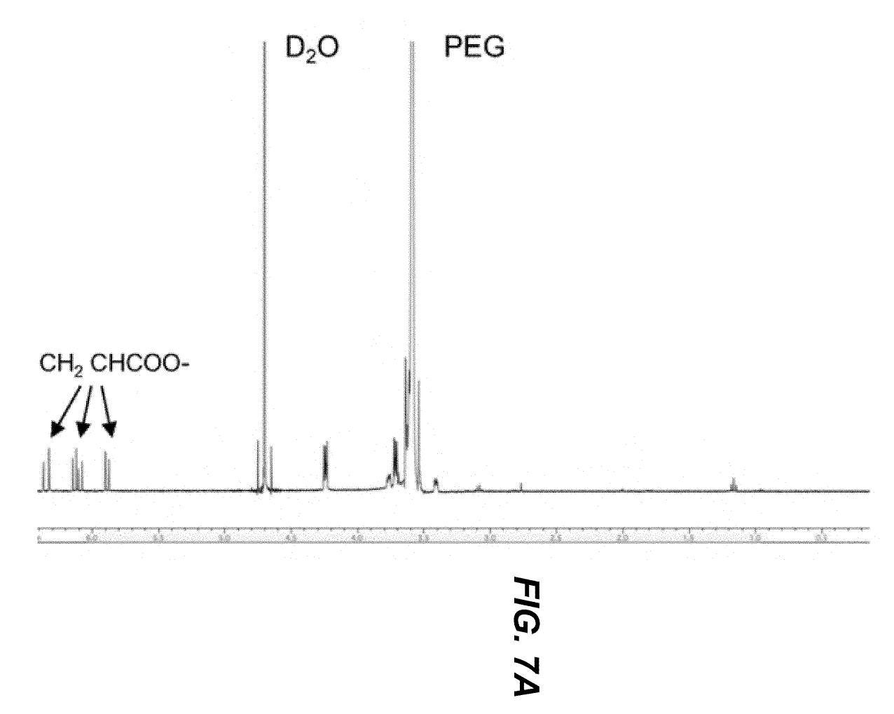

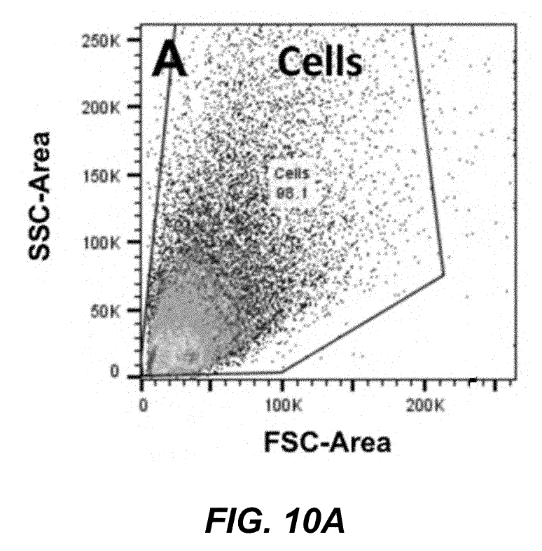

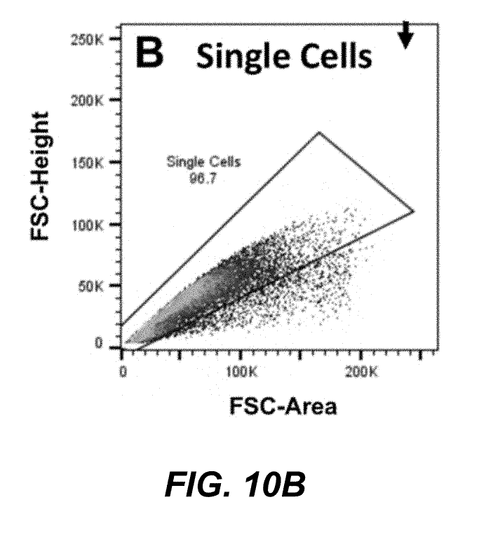

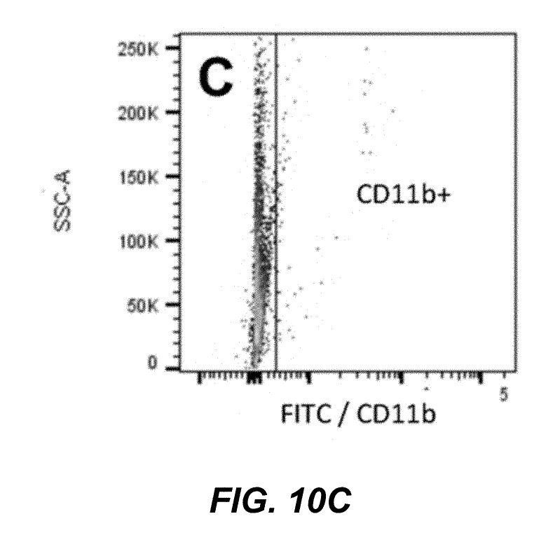

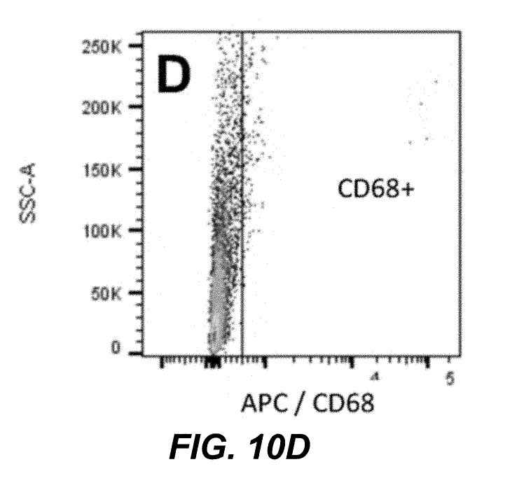

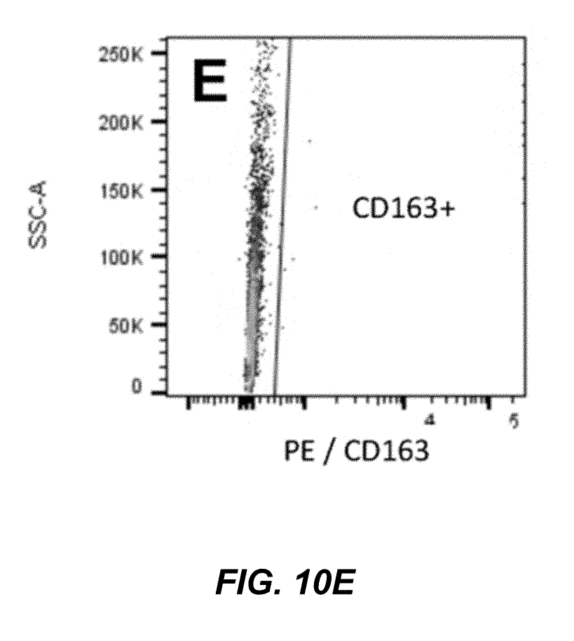

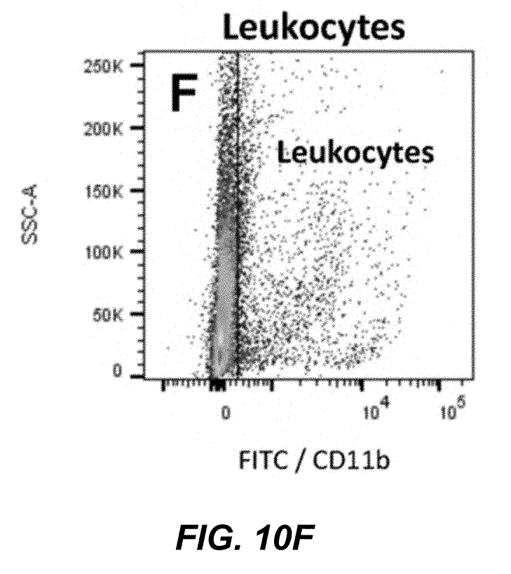

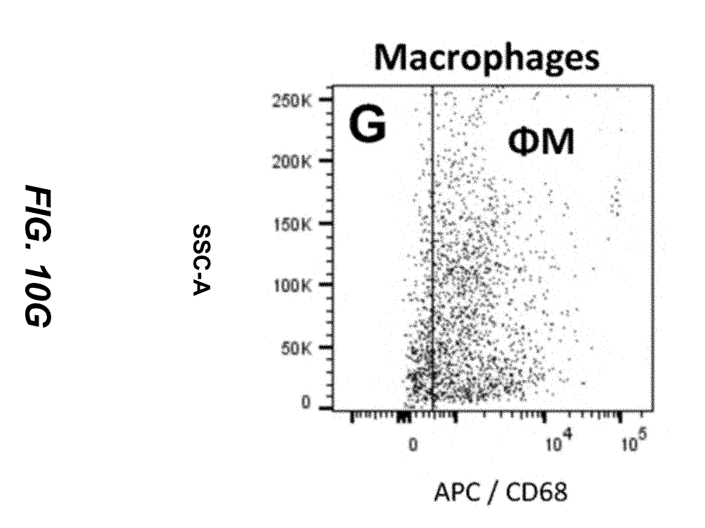

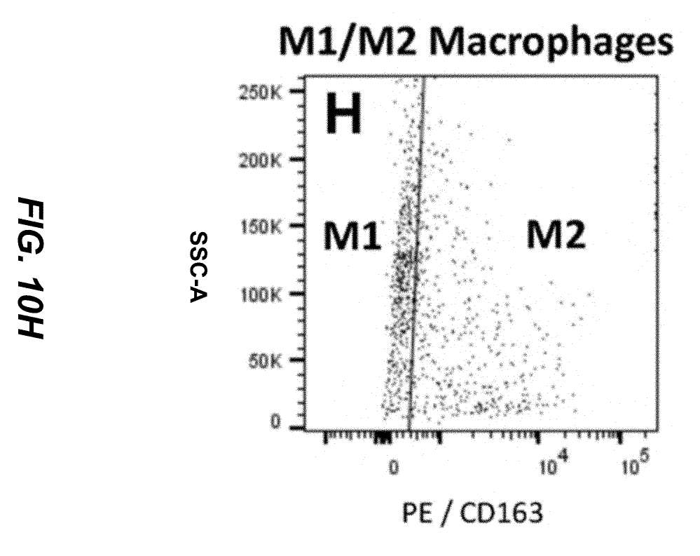

Supraspinatus muscles were analyzed for mesenchymal cell (MSC) infiltration via flow cytometry at days 3 and 7. Similar to the trends seen in inflammatory cell analysis, there were no significant differences in MSC recruitment between any experimental group or between each experimental group and the uninjured contralateral controls at day 3 (1.1-1.4×, FIG. 5A). However, by day 7 there were significantly more MSCs in both the SDF-1α loaded MP group (3.0±0.8×) and the unloaded MP group (1.7±0.6×) compared to their respective uninjured contralateral controls, and significantly more MSCs were recruited to the SDF-1α loaded MP group than unloaded MPs and injury alone (FIG. 5B). Flow cytometry data are shown in FIGS. 10(A-H). Example data from supraspinatus muscle shows the identification of FIG. 10(F) leukocytes, CD11b+, FIG. 10(G) macrophages, CD11b+CD68+, and FIG. 10(H) M1 or M2 macrophages, CD11b+CD68+CD163−/+.

### Example 12

### Vascular Staining of Whole-Mounted Supraspinatus Muscle

After 7 days following injury and treatment, supraspinatus muscles were fixed in 4% PFA for 30 minutes at RT, rinsed in PBS, permeabilized in 0.2% saponin (Sigma) PBS solution for 24 hours at 4° C., and blocked in 10% BSA solution for 24 hours at 4° C. For vascular staining, muscles were incubated in 5 μg/mL anti-mouse/rat CD31/PECAM-1 primary antibody (R&D Systems) in a 1.0% BSA, 0.3% Triton X-100 (Amresco), 0.01% sodium azide solution (incubation buffer) overnight at 4° C., followed by four 30-minute washes in 0.2% saponin solution. Muscles were then stained in a 1:200 dilution of NL557-conjugated anti-goat IgG secondary antibody (R&D Systems) in incubation buffer for 4 hours at RT, then washed in 0.2% saponin solution and PBS twice each for 30 minutes. As a negative control, samples were stained using the same protocol but with polyclonal goat IgG isotype control (R&D Systems) in place of the primary antibody. Finally, muscles were incubated in a 1:1000 dilution of Hoechst cellular stain in PBS for 5 minutes. Muscles were whole-mounted and single fluorescent images were obtained using a Zeiss LSM 700 confocal microscopy with a 10× objective to visualize vasculature (green) within muscle tissue (blue); n=2 animals per group.

### Example 13

### Statistical Analysis

One-way analysis of variance (ANOVA) and Tukey's post hoc multiple comparison test with a significance value set at p≤0.05 were used to identify significant differences. Statistical analysis was performed with Prism software. Data throughout the examples are presented as mean±standard deviation.

### Example 14

### Materials Characterization

Using ¹H NMR analysis, PEGDA was determined to be ˜55% functionalized (FIG. 7A) and Hep methacrylamide was determined to be 22-28% functionalized (FIG. 7B). SAX-HPLC analysis was also used to assess Hep and Hep^{-N} disaccharide composition. Disaccharide elution patterns showed evidence of N-desulfation when comparing Hep^{-N} against Hep samples (Table 1). Finally, using SEC-HPLC, the weight average molecular weight was determined to be ˜13.5 kDa for Hep^{-N} compared to ˜18.4 kDa for Hep.

TABLE 1
Heparin and N-desulfated heparin disaccharide
composition as determined by SAX-HPLC analysis.

| | ΔUA(2S)- | ΔUA(2S)- | ΔUA- | ΔUA- | ΔUA(2S)- | ΔUA(2S)- | ΔUA(2S)- |
|---|---|---|---|---|---|---|---|
| | GlcNS(6S) | Glc(6S) | GlcNS(6S) | Glc(6S) | GlcNS | Glc | GlcNAc(6S) |
| Hep | 74 | ND | 8 | ND | 6 | ND | 2 |
| Hep^{-N} | ND | 72 | ND | 10 | ND | 7 | ND |

ΔUA = Δ^{4,5}-unsaturated uronic acid; Glc = glucosamine; NS = N-sulfo; NAc = N-acetyl; ND = not detected.

### Example 15

### Microparticle Fabrication and Degradation Study

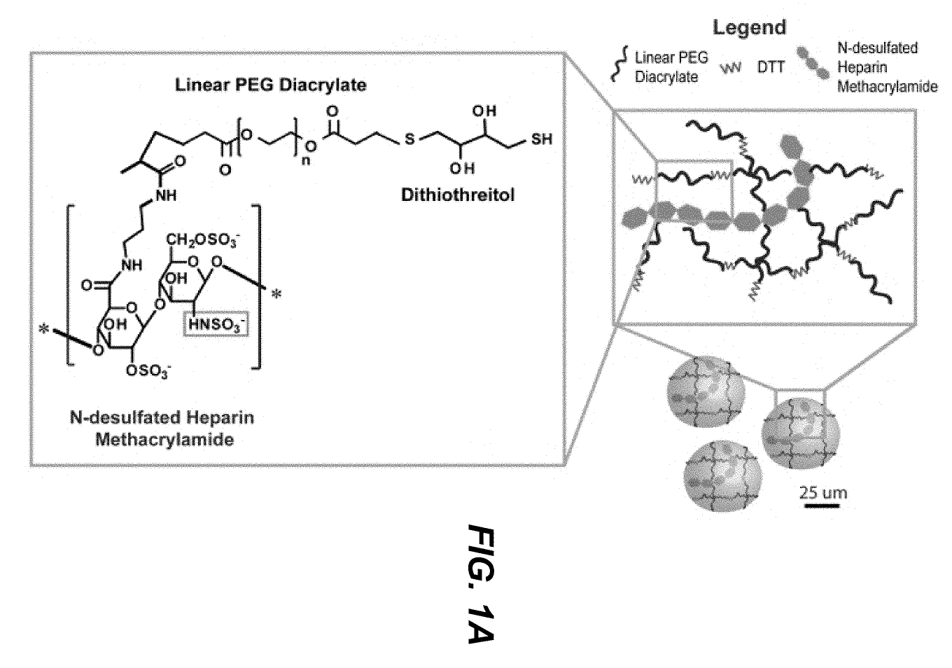

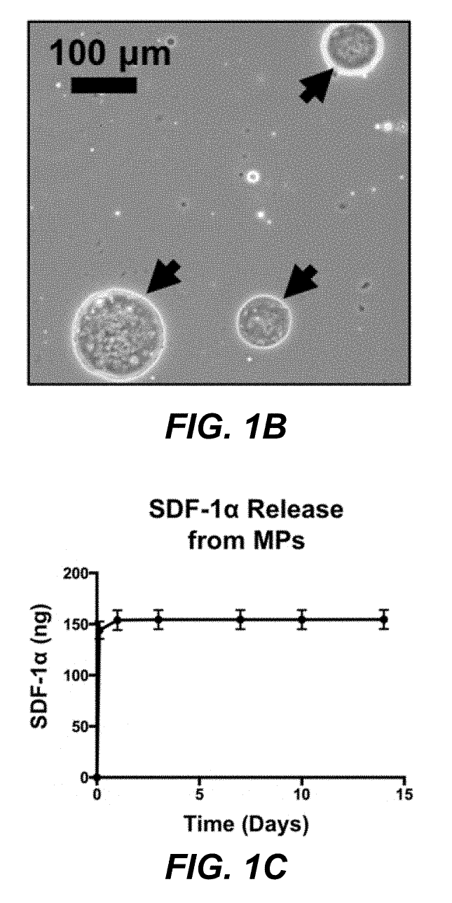

Degradable 10 wt % Hep^{-N} MPs were found to be 62±65 μm in diameter (FIG. 1B). The degradable particles were monitored for degradation in vitro by incubating 0.5 mg MPs in 0.5 mL 0.1 wt % BSA solution at 37° C. 30 μL of each sample was removed and imaged via phase microscopy every two days until complete degradation; n=3 per MP formulation. MP degradation was defined as the time point at which no MP were found across the entire microscope slide, at which point no image was taken. Additionally, by varying DTT concentration within MPs, MP degradation ranged between 30 days for 20 mM DTT to 8 days for 40 mM DTT (FIG. 8). MPs used in all subsequent studies contained 35 mM DTT, which degraded within 16 days in vitro (FIG. 8).

### Example 16

### SDF-1α Loading and Release from Microparticles

For all MP batches, 155±10 ng SDF-1α was released from MPs over 7 days in vitro (FIG. 1C). Once ˜155 ng SDF-1α was determined to be an effective dosage from pilot studies in vivo, cumulative release of SDF-1α from MPs was held constant across all in vivo experiments by varying the initial mass of SDF-1α added to each batch of MPs between 1.0-1.2 SDF-1α.

### Example 17

### In Vivo Injection of Microparticles

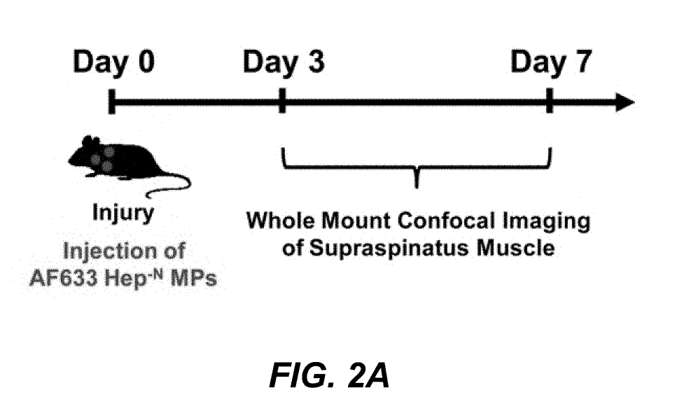

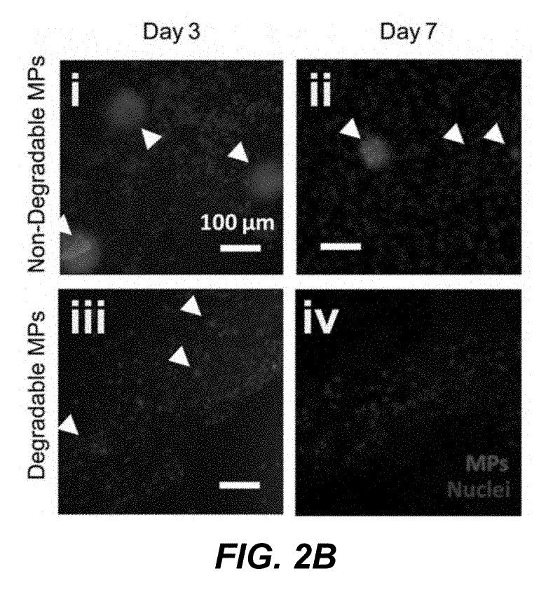

Intact, non-degradable AF633 Hep^{-N} MPs (red) were visible within the supraspinatus muscle (blue) 3 and 7 days following injection (FIGS. 2Bi-2Bii), whereas degradable AF633 Hep^{-N} MPs were present at day 3 but no longer detectable by day 7 (FIG. 2Biii).

### Example 18

### Muscle Degeneration After Rotator Cuff Injury

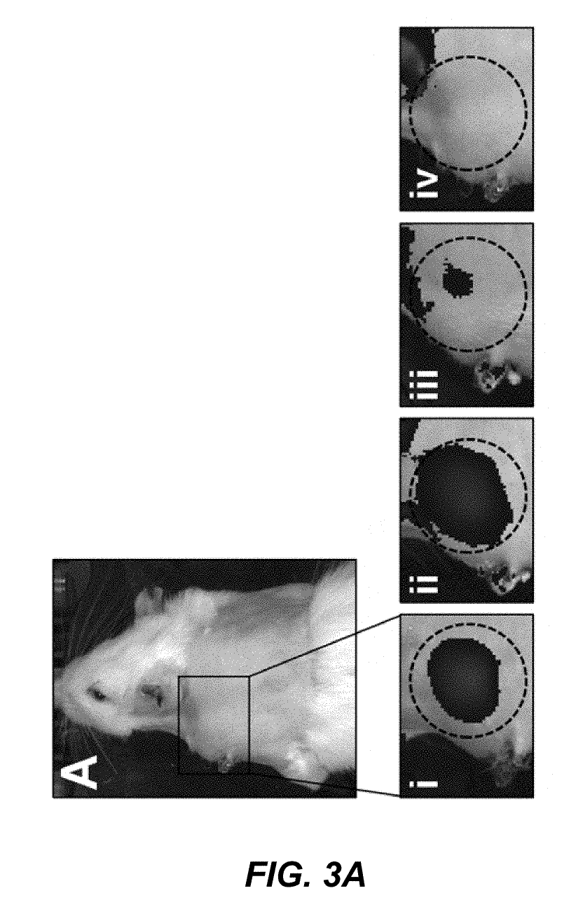

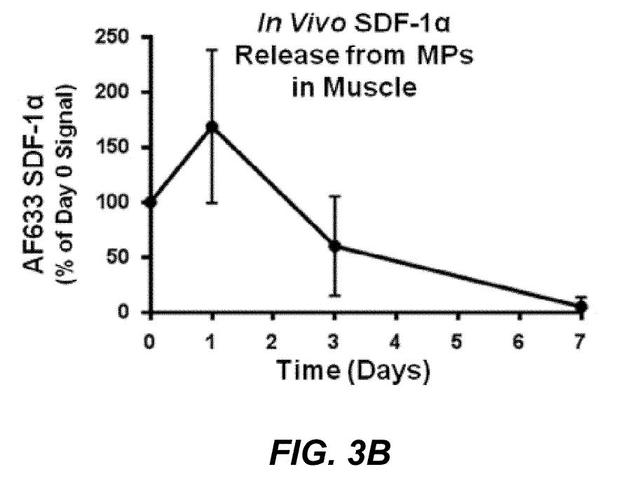

Supraspinatus muscle weight significantly decreased by 40±9% compared to uninjured controls at week 6 (FIG. 3A). Masson's trichrome showed that fibrous infiltration was significantly greater in the injured supraspinatus muscles as compared to uninjured controls at both the 3 and 6 week time points (FIGS. 3B-3C).

### Example 19

### Vascular Staining of Whole-Mounted Supraspinatus Muscle

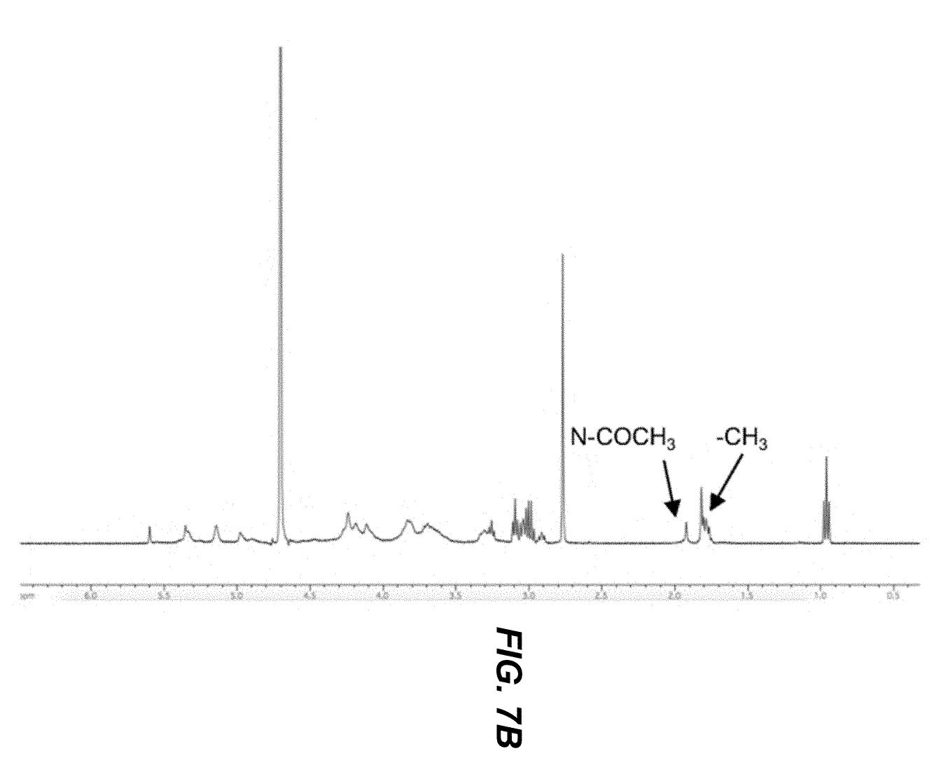

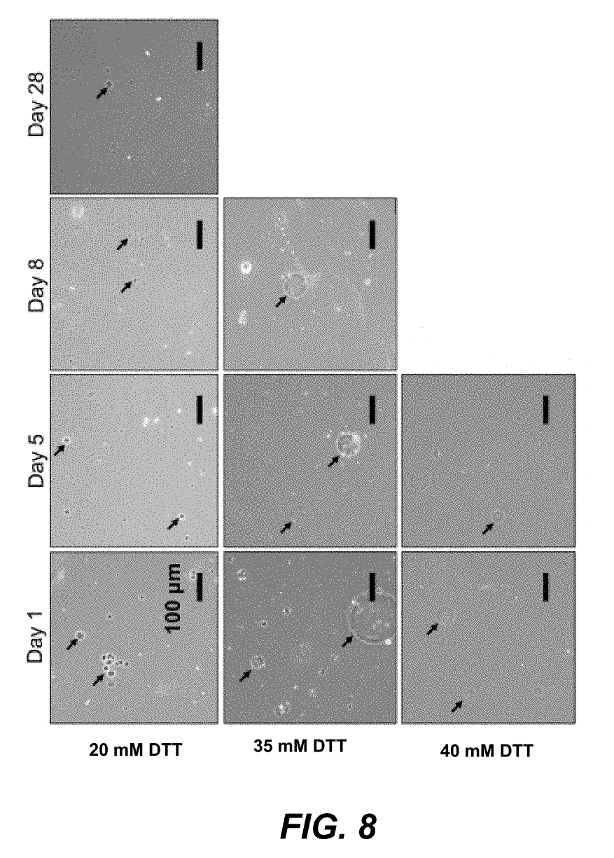

Vascular staining of whole-mounted supraspinatus muscle was undertaken. Compared to uninjured contralateral controls that had little CD31+ vascular staining (FIG. 6A), CD31+ vascular looping, a product of rapid angiogenesis whereby vessels elongate and form loops of vasculature, appeared to be present in muscles treated with SDF-1α loaded MPs (FIG. 6B). No staining was observed in samples prepared with isotype controls.

### Example 20

### Heparin Modifications

N-desulfated (Hep^{-N}) and fully desulfated (Hep−) heparin were prepared by dissolving 10 mg/mL heparin sodium salt (Hep) from porcine intestinal mucosa (Sigma) in dH₂O and passing through Dowex 50WX4 resin (mesh size 100-200, Sigma). Tellier L. E. et al., J Mater Chem B. 2015, and Seto S. P. et al., Bioconjug Chem. 2015; 26(2):286-293. doi:10.1021/bc500565x. Pyridine was added until the heparin solution reached pH 6, after which time excess dH₂O and pyridine were removed via rotatory evaporator (Buchi), flash frozen, and lyophilized. For Hep^{-N}, the heparin pyridinium was then dissolved at 1 mg/mL in 9:1 v/v dimethyl sulfoxide (DMSO)/dH₂O at 50° C. for 2 hours. Inoue Y. et al., Carbohydr Res. 1976; 46:87-95, and Nagasawa K. et al., Carbohydr Res. 1977; 58:47-55. For Hep−, heparin pyridinium was dissolved at 10 mg/mL in 9:1 v/v N-methylpyrrolidone (NMP, Acros Organics)/dH₂O at 100° C. for 24 hours. Baumann H. et al., Carbohydr Res. 1998; 308:381-388. Subsequently, Hep^{-N} and Hep− were precipitated with 95% ethanol saturated with sodium acetate, collected via centrifuge, dissolved in dH₂O, dialyzed, lyophilized, and stored at −20° C.

Hep^{-N} methacrylamide (Hep^{-N} MAm) functionalization was performed as described previously. Rinker T. E. et al., Acta Biomater. 2016. Briefly, 1.1 mM Hep^{-N}, 83.0 mM N-hydroxysulfosuccinimide (sulfo-NHS, Sigma), 101.0 mM N-(3-Aminopropyl) methacrylamide hydrochloride (APMAm, Polysciences Inc.), and 156.0 mM (N-3-Dimethylaminopropyl)-N′-ethylcarbodiimide hydrochloride (EDC, Sigma) were combined in 10 mL phosphate buffer saline (PBS, Teknova). After stirring on ice for 6 hours, Hep^{-N} MAm was dialyzed, lyophilized, and stored at −20° C.

### Example 21

### Heparin Modifications

To fabricate 10 wt % Hep MPs, 50.0 mg PEGDA and 1.61 mg dithiothreitol (DTT, 35 mM, Sigma) were added to 273 μL 10 wt % BSA PBS solution and incubated at 37° C. for 30 minutes to allow for Michael Type addition between PEGDA and DTT. Then, 5.6 mg Hep^{-N}MAm was added and the aqueous solution was incubated again at 37° C. for 30 minutes.

Next, an oil phase of 5 mL mineral oil (Amresco) with 3.0-3.2 μL Span80 (TCI) was placed under a homogenizer (Polytron PT 3100, Kinematica) set to 4000 RPM. After adding 27 μL of 0.05 wt % Irgacure 2959 photoinitiator (Ciba) to the aqueous phase, the solution was added drop-wise to the oil phase and the water-and-oil emulsion was allowed to homogenize for 5 minutes. Subsequently, the water-and-oil emulsion was nitrogen purged for 1 minute and crosslinked under UV (˜15 mW/cm²) via free radical polymerization between PEGDA and Her^{-N} MAm. MPs were then washed through 3 iterations of the following procedure: MPs were combined with 35 mL dH₂O, centrifuged at 4000 RPM for 5 minutes, and the supernatant consisting of water and oil was removed. In the final wash, MPs were pipetted through 40 μm cell strainers to remove MPs under 40 μm in diameter. To sterilize MPs, each MP batch was incubated with 70% ethanol on rotary platform at 4° C. for 30 minutes, followed by three 30-minute washes in sterile PBS. Phase microscopy and ImageJ software were used to image and determine the size distribution of each MP batch. MPs were stored in sterile PBS at 4° C. until use.

### Example 22

### TSG-6 Loading and Release from Microparticles

To load TSG-6 onto MPs, 1.0 μg human TSG-6 (R&D Systems) was added to 0.6 mg MPs in 50 μL 0.1 wt % BSA solution. TSG-6 and MPs were incubated for 2 hours at 4° C., after which time MPs were rinsed by adding an additional 450 μL 0.1 wt % BSA solution. The MPs were centrifuged for 3 minutes at 10,000 RCF and 495 μL supernatant was removed.

For in vitro TSG-6 release studies, the removed supernatant was replaced with 495 μL fresh 0.1 wt % BSA solution and samples were incubated at 37° C. MPs were centrifuged and 495 μL supernatant was removed and replaced 3 hours, 1, 3, 7, and 10 days following TSG-6 loading until MPs degraded. TSG-6 protein levels were quantified using a human TSG-6 sandwich ELISA with the following steps: 10 μg/mL monoclonal capture antibody (Santa Cruz Biotechnology) overnight, 100-10,000 pg/mL recombinant human TSG-6 used as standards and samples for 2 hours, 0.5 μg/mL biotinylated secondary detection antibody (R&D Systems) for 2 hours, streptavidin-horseradish peroxidase enzyme (R&D Systems) for 20 minutes, substrate solution (R&D Systems) for 20 minutes and lastly a stop solution of 2 N sulfuric acid (Ricca) that was read at 450 nm; n=3-5.

To assess TSG-6 bioactivity after release from MPs, the plasmin inhibition assay protocol was followed with day 1 TSG-6 release supernatant (11 ng/mL) and compared to a soluble TSG-6 control at the same concentration (11 ng/mL); n=3-5.

### Example 23

### Medial Meniscal Transection Model

Medial meniscal transection (MMT) injury was induced using a similar method to previously established protocols. Willett N. J. et al., Arthritis Res Ther. 2014; 16(1):R47. Male Sprague-Dawley rats (250-300 g initial weight and 8-10 weeks old) were used in accordance with protocols approved by the Georgia Institute of Technology Institutional Animal Care and Use Committee. Prior to surgery animals were anesthetized by 5% isoflurane (Isothesia), followed by 2-3% isoflurane during surgery and were administered sustained release buprenorphine as an analgesic. Next, a small incision was made through the skin on the medial aspect of the left femoral-tibial joint. The medial collateral ligament was exposed by blunt dissection and transected to visualize the joint space and medial meniscus. The meniscus was then transected completely at its narrowest point. The skin was sutured with 4.0 silk sutures (Ethicon) and then closed using wound clips.

### Example 24

### In Vivo TSG-6 Delivery

For TSG-6 loaded MP treatment, MPs were loaded as described above but 3.6 mg MPs were resuspended in a total volume of 50 μL sterile 0.1 wt % BSA PBS solution and subsequently loaded into sterile syringes. 1, 7, and 15 days following MMT injury, MPs were delivered via intra-articular injection through the infrapatellar ligament and into the stifle joint of the left leg. For soluble TSG-6 controls, 16.7 μg TSG-6 was dissolved in a total volume of 50 μL sterile 0.1 wt % BSA PBS solution, loaded into sterile syringes, and delivered using the same method as TSG-6 loaded MPs. Overall, in vivo experimental groups included TSG-6 loaded MPs (18 μg TSG-6 loaded onto 10.8 mg MPs delivered over 3 time points; n=3), soluble TSG-6 (50 μg TSG-6 delivered over 3 time points; n=3) or injury only (no TSG-6 or MPs; n=4). Contralateral tibiae served as uninjured controls.

### Example 25

### MicroCT Analysis

21 days following MMT injury, rats were euthanized and tibiae were harvested and fixed in 10% neutral buffered formalin (EMD Chemicals) for 7 days. Tibiae were then immersed in a 30% Hexabrix (Covidien) PBS solution at 37° C. for 30 minutes and scanned using a μCT40 (Scanco Medical) at 45 kVp, 177 mA, and 200 ms integration time.

For cartilage assessment via μCT, Equilibrium Partitioning of an Ionic Contrast agent-μCT (EPIC-μCT) was used as described previously. Xie L. et al., Osteoarthr Cartil. 2009; 17(3):313-320. Scanco evaluation software was used to measure cartilage thickness, volume, attenuation, surface roughness, and lesion volume within the medial third of the medial tibial plateau, which is the characteristic region of damage in MMT injuries. Willett N. J. et al., Arthritis Res Ther. 2014; 16(1):R47, and Thote T. et al., Osteoarthr Cartil. 2013; 21(8):1132-1141. Raw scan data were automatically reconstructed to 2D grayscale tomograms, which were subsequently rotated to sagittal sections. Cartilage was contoured to separate it from trabecular bone and surrounding area. The cartilage was segmented using a bandpass filter with a minimum threshold value of 75 to eliminate air and 355 to eliminate bone from the raw image following which 3D images were generated. The threshold values were globally applied for both the left and right tibiae of all animals; n=3-4.

### Example 26

### Histology

Following μCT, tibiae were decalcified in Cal-Ex II (Fisher) for 10 days, then processed for histology sectioning as described previously. Tellier L. E. et al. Localized SDF-1 Delivery Increases Pro-Healing Bone Marrow-Derived Cells in the Supraspinatus Muscle Following Severe Rotator Cuff Injury. Acta Biomater. 2017. Sections were stained in 0.1% fast green (Sigma) and 0.25% safranin-O solution (Sigma) and imaged at 20× magnification with a Nikon Eclipse 80i; n=3-4.

### Example 27

### Statistical Analysis

All data are presented as mean±standard deviation. One-way analysis of variance (ANOVA) and Tukey's post hoc multiple comparison test (significance value of p≤0.05) were run using Prism software.

### Example 28

### Materials Characterization

¹H NMR indicated that PEGDA was ˜55% functionalized while Hep^{-N} MAm was between 22-28% functionalized. Percent modification was determined by dividing the integral of the methacrylamide peak by the heparin peak for Hep^{-N} MAm and the acrylate peaks by the PEG peak for PEGDA.

### Example 29

### Plasmin Inhibition Assay with Soluble Heparin Derivatives

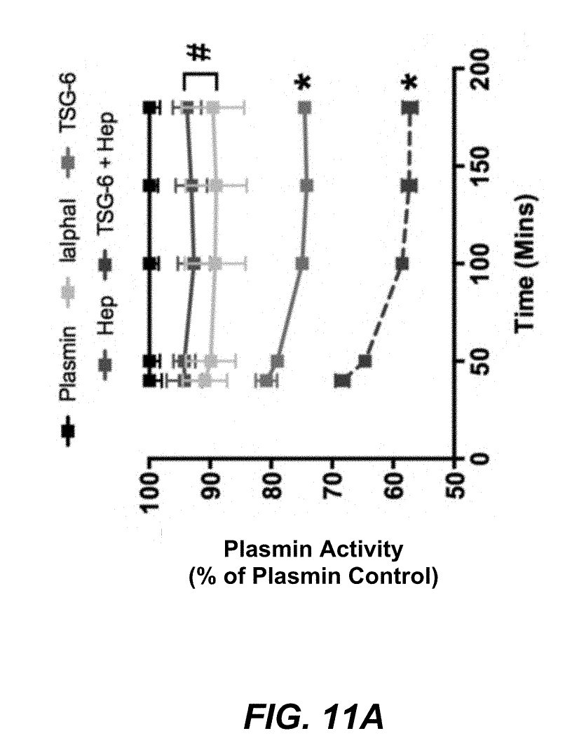

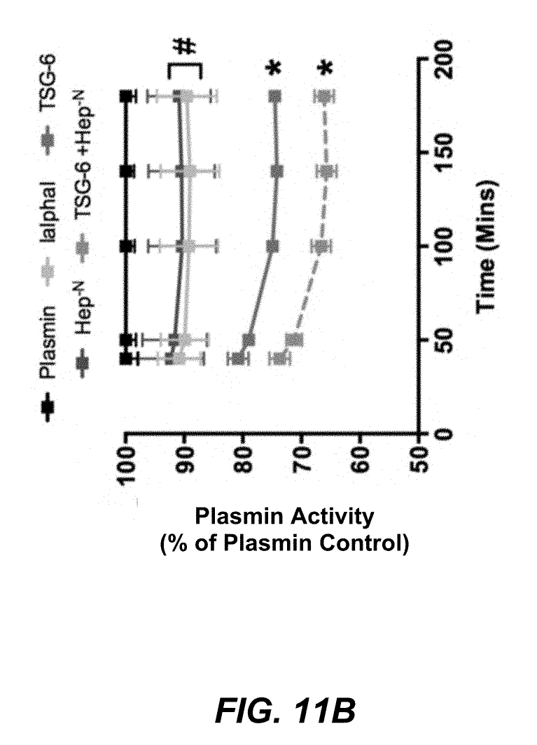

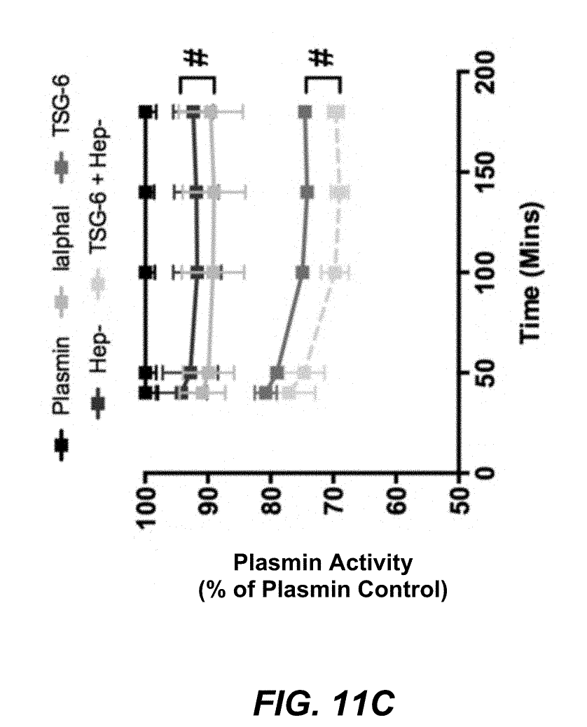

Hep and Hep^{-N} both significantly enhanced TSG-6 anti-plasmin activity (57.3±1.2% and 66.1±1.7% plasmin activity compared to plasmin control, respectively) compared to TSG-6 alone (74.6±0.2%), though Hep enhanced TSG-6 activity significantly more than Hep^{-N}. In contrast, Hep− had no significant effect (69.6±1.2%) compared to TSG-6 alone (FIG. 11). Without wishing to be bound by theory, it may be that the sulfate groups removed, the N sulfate group for Hep^{-N} and the N and 20 sulfate groups for Hep−, are necessary for complete heparin binding to TSG-6 and the subsequent enhancement of TSG-6 bioactivity. In addition, more sulfated heparin derivatives have also been shown to protect proteins from denaturation, providing an additional mechanism by which Hep and Hep^{-N} may have maintained TSG-6 bioactivity. Further, heparin sulfation may play a significant role in modulating TSG-6 bioactivity because sulfated heparin derivatives (Hep and Hep^{-N}) significantly enhanced TSG-6 anti-plasmin activity in vitro, whereas fully desulfated heparin (Hep−) had no effect.

### Example 30

### Microparticle Fabrication, Loading and Release

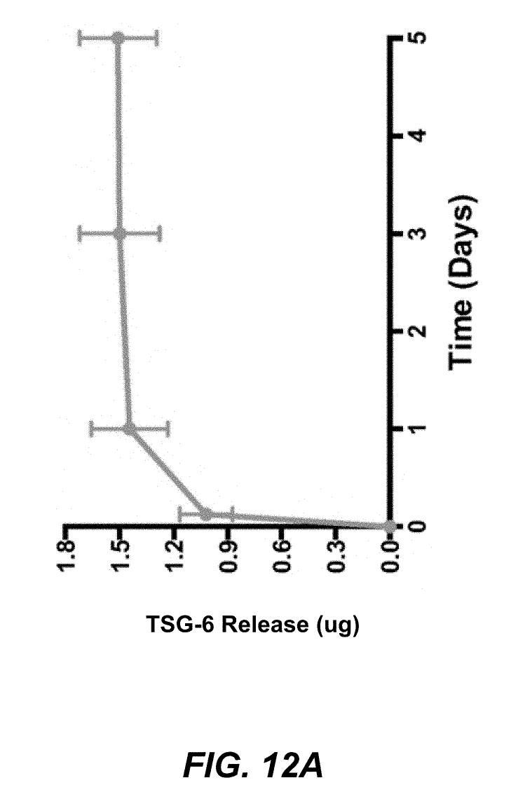

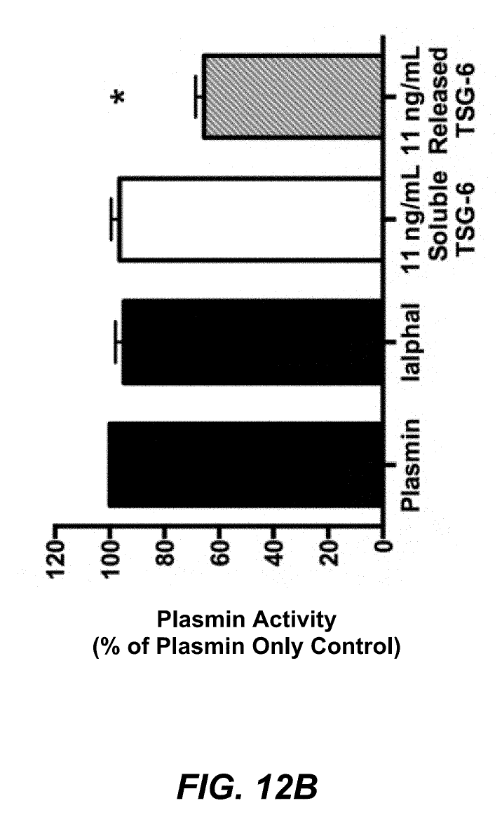

Degradable 10 wt % Hep^{-N} MPs were found to be 80±60 μm in diameter (FIG. 12) and have previously been shown to degrade within 10-16 days in vitro. Tellier L. E. et al. Localized SDF-1 Delivery Increases Pro-Healing Bone Marrow-Derived Cells in the Supraspinatus Muscle Following Severe Rotator Cuff Injury. Acta Biomater. 2017. In vitro, 6.0 μg TSG-6 was loaded onto MPs and over 5 days ˜1 μg TSG-6 was released (FIG. 12A). Therefore, over 3 injections in vivo, 18.0 μg TSG-6 was originally added to MPs and ˜4.5 μg TSG-6 was released. In comparison, for soluble TSG-6 treatment, 16.7 μg TSG-6 was delivered per injection, resulting in a total dosage of 50.0 μg soluble TSG-6 over three injections. Importantly, TSG-6 released from MPs after 1 day exhibited significantly greater anti-plasmin activity (65.7±3.0% plasmin activity compared to plasmin control) than soluble TSG-6 (96.4±3.1%) at the same concentration (FIG. 12B).

### Example 31

### MicroCT Analysis

To determine the ability for Hep^{-N}-based MPs to enhance TSG-6 treatment in vivo, the effect of soluble TSG-6 and injury alone was first assessed via EPIC-μCT, an analysis technique which utilizes a contrast agent to better distinguish cartilage from bone in μCT images. Xie L. et al., Osteoarthr Cartil. 2009; 17(3):313-320, and Palmer A. W. et al., Proc Natl Acad Sci USA. 2006; 103(51):19255-19260.

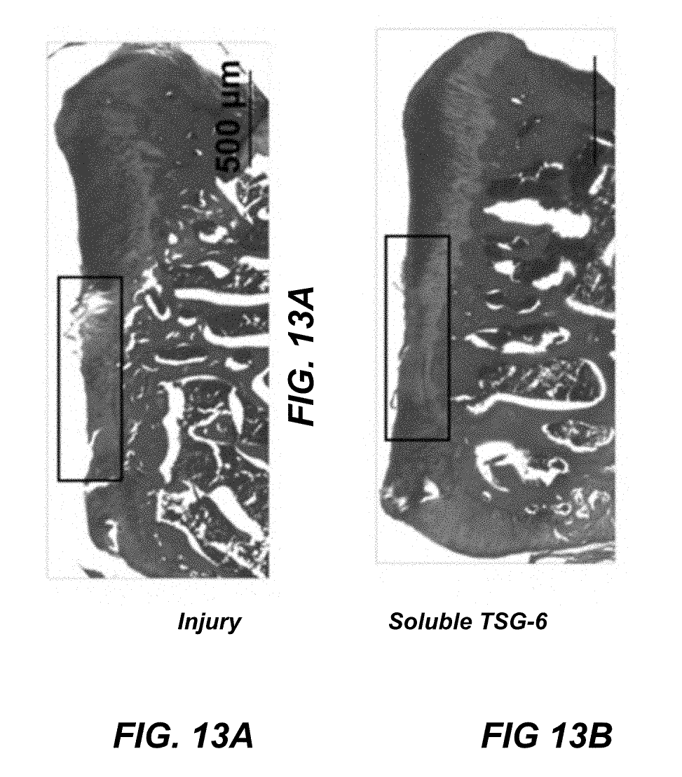

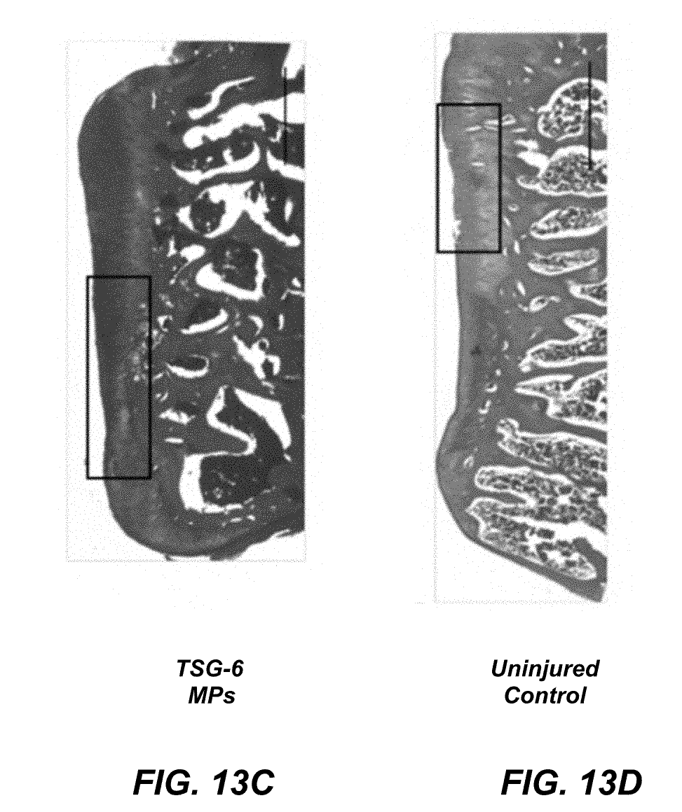

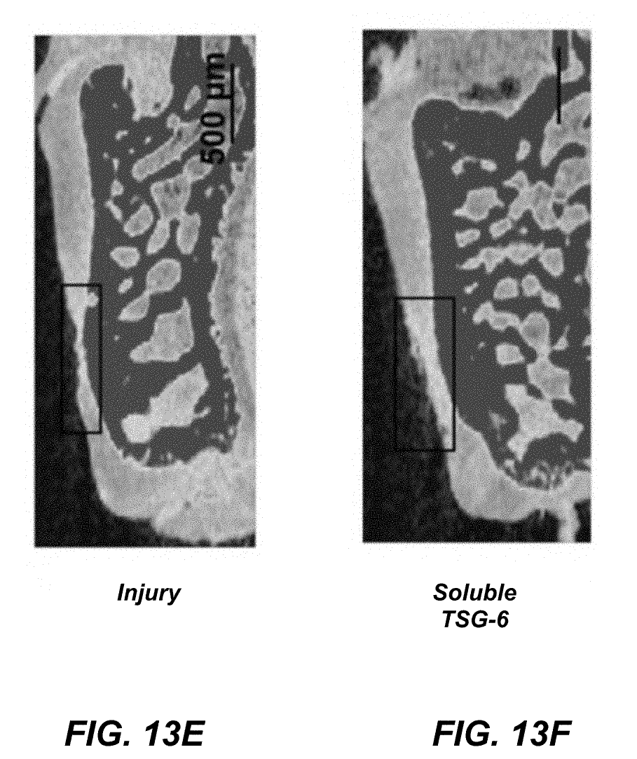

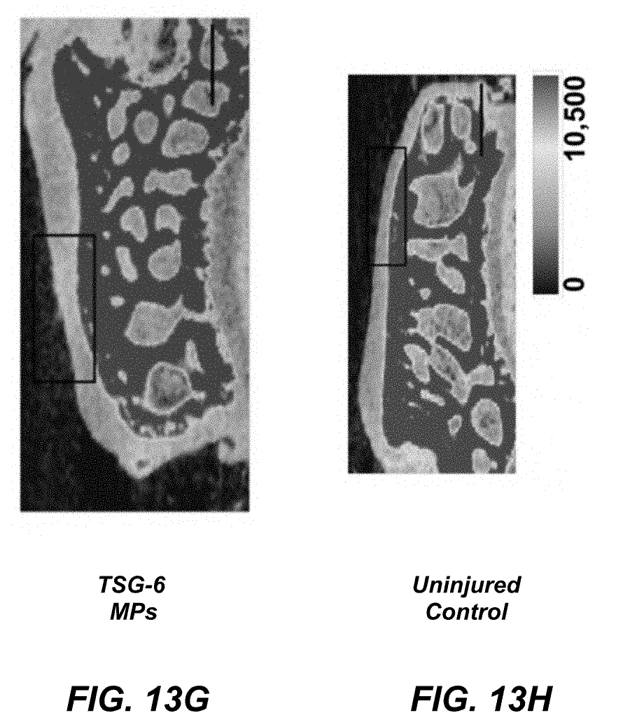

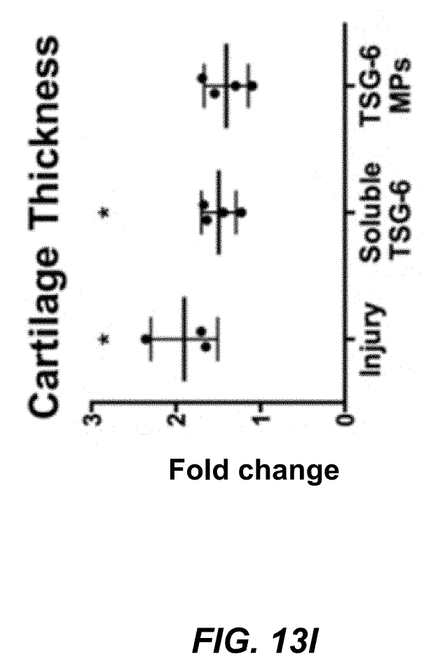

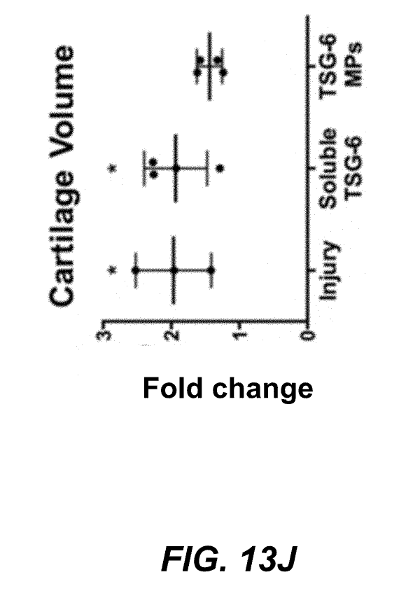

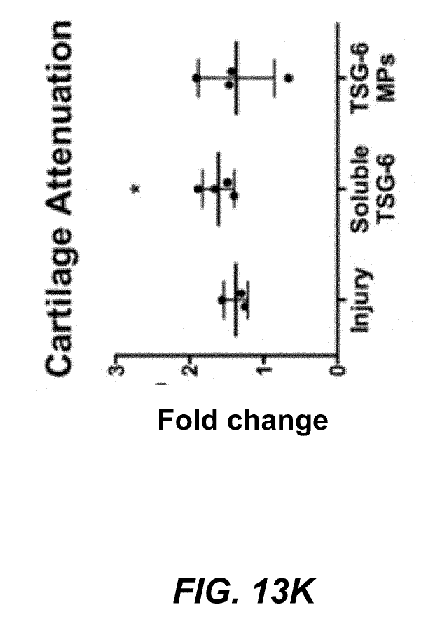

μCT images 21 days following MMT injury were quantified to assess changes in cartilage thickness, volume, and attenuation (FIGS. 13E-13H). After 21 days following MMT injury, significantly increased cartilage thickness (1.9±0.4× compared to uninjured control) and volume (2.0±0.6×) was observed compared to uninjured contralateral controls (FIG. 13I-J). Soluble TSG-6 treated samples also exhibited significantly increased cartilage thickness (1.5±0.2×) and volume (1.9±0.5×) as well as increased attenuation (1.6±0.2×) (FIGS. 13I-13K). In contrast, neither cartilage thickness, volume, nor attenuation were increased in the TSG-6 loaded MP group compared to uninjured controls (FIGS. 13I-13K). Osteophyte volume, focal lesion volume, and surface roughness were not significantly different between each experimental group or compared to respective contralateral controls (data not shown).

In contrast to injury and soluble TSG-6, EPIC-μCT revealed that neither cartilage thickness, volume, nor attenuation were increased in the TSG-6 loaded MP group compared to uninjured controls (FIGS. 13G and 13I-K) and histological analysis showed that cartilage safranin-O staining remained similar to uninjured controls, indicating that cartilage hypertrophy and proteoglycan loss were reduced by TSG-6 loaded MP treatment (FIG. 13C). Hep^{-N} can enhance TSG-6 bioactivity in vivo.

### Example 32

### Histology

Safranin-O staining of GAG within cartilage was observed in TSG-6 loaded MP treated tibiae (FIG. 13C) and in uninjured controls (FIG. 13D). Less intense safranin-O staining was observed in the medial one-third of the injured and soluble TSG-6 treated tibiae (FIGS. 13A-13B). Soluble TSG-6 was unable to improve cartilage degeneration following injury (FIG. 13B). In contrast, improvement was seen with the TSG-6 loaded MPs prepared as above (FIGS. 13C).

### Example 33

### Small Molecule Loading and Release from Microparticles

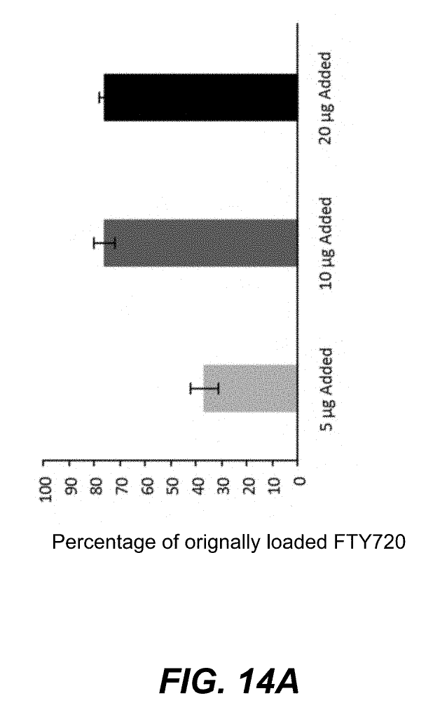

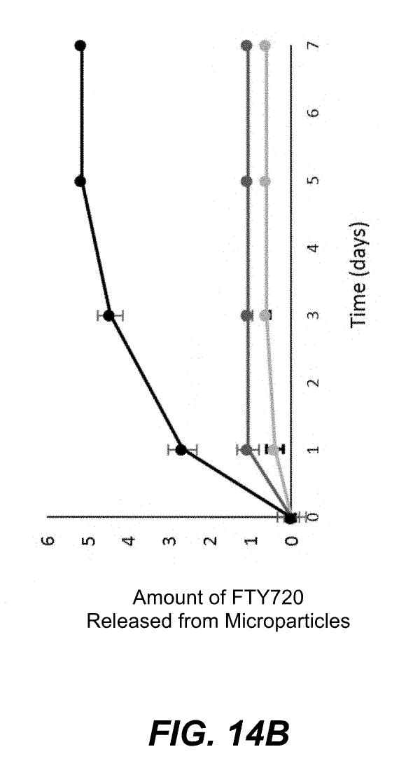

FTY720 was loaded onto degradable heparin-based MPs. The amount of FTY720 that was loaded onto the MPs was then assayed via plate reader. In three separate groups, additional amounts of FTY720, i.e. 5 μg, 10 μg and 20 μg, were then added. FIG. 14(A) shows the loading efficiency of the small molecule FTY720 varying between 35-80% of the originally added amount of FTY720. For each of the three separate groups, the extent of FTY720 release was assayed via plate reader each day over 7 days. FIG. 14(B) shows the total amount of FTY720 released from MPs varying between about 0.5-5 μg over 7 days dependent on the amount of FTY720 originally added to MPs. The black line refers to the batch with 20 μg added. The dark blue line refers to the batch with 10 μg added. The light blue line refers to the batch with 5 μg added.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

### REFERENCES

1. Yamaguchi K, Ditsios K, Middleton W D, Hildebolt C F, Galatz L M, Teefey S A. The Demographic and Morphological Features of Rotator Cuff Disease: A Comparison of Asymptomatic and Symptomatic Shoulders. J Bone Jt Surg. 2006; 88(8):1699-1704. doi:10.2106/JBJS.E.00835.
2. Gladstone J N, Bishop J Y, Lo I K Y, Flatow EL. Fatty Infiltration and Atrophy of the Rotator Cuff Do Not Improve After Rotator Cuff Repair and Correlate With Poor Functional Outcome. Am J Sports Med. 2007: 719-728. doi:10.1177/0363546506297539.
3. Gerber C, Fuchs B, Hodler J. The results of repair of massive tears of the rotator cuff. J Bone Joint Surg Am. 2000; 82(4):505-515.
4. Chargé S B P, Rudnicki M. Cellular and molecular regulation of muscle regeneration. Physiol Rev. 2004; 84(1):209-238. doi:10.1152/physrev.00019.2003.
5. Karalaki M, Fili S, Philippou A, Koutsilieris M. Muscle regeneration: cellular and molecular events. In Vivo. 2009; 23(5):779-796. doi:23/5/779 [pii].
6. Roche J A, Tulapurkar M E, Mueller A L, et al. Myofiber damage precedes macrophage infiltration after in Vivo injury in dysferlin-deficient A/J mouse skeletal muscle. Am J Pathol. 2015; 185(6):1686-1698. doi:10.1016/j.ajpath.2015.02.020.
7. Ogle M E, Segar C E, Sridhar S, Botchwey E A. Monocytes and macrophages in tissue repair: Implications for immunoregenerative biomaterial design. Exp Biol Med. 2016; 241(10):1084-1097. doi:10.1177/1535370216650293.
8. Tidball J G. Inflammatory processes in muscle injury and repair. Am J Physiol—Integr Comp Physiol. 2005; 288:345-353. doi:10.1152/ajpregu.00454.2004.
9. Chazaud B, Brigitte M, Yacoub-Youssef H, et al. Dual and beneficial roles of macrophages during skeletal muscle regeneration. Exerc Sport Sci Rev. 2009; 37(1):18-22. doi:10.1097/JES.0b013e318190ebdb.
10. Arnold L, Henry A, Poron F, et al. Inflammatory monocytes recruited after skeletal muscle injury switch into antiinflammatory macrophages to support myogenesis. J Exp Med. 2007; 204(5):1057-1069. doi:10.1084/jem.20070075.
11. Massimino M L, Rapizzi E, Cantini M, et al. ED2+ macrophages increase selectively myoblast proliferation in muscle cultures. Biochem Biophys Res Commun. 1997; 235(3):754-759. doi:10.1006/bbrc.1997.6823.
12. Deng B, Wehling-Henricks M, Villalta S A, Wang Y, Tidball J G. IL-10 Triggers Changes in Macrophage Phenotype That Promote Muscle Growth and Regeneration. J Immunol. 2012; 189(7):3669-3680. doi:10.4049/jimmuno1.1103180.
13. Tidball J G, Wehling-henricks M. Macrophages promote muscle membrane repair and muscle fibre growth and regeneration during modified muscle loading in mice in vivo. J Physiol. 2007; 1:327-336. doi:10.1113/jphysiol.2006.118265.
14. Merly F, Lescaudron L, Rouaud T, Crossin F, Marie France Gardahaut. Macrophages enhance muscle satellite cell proliferation and delay their differentiation. Muscle Nerve. 1999; (June):724-732.
15. Peng H, Huard J. Muscle-derived stem cells for musculoskeletal tissue regeneration and repair. Transpl Immunol. 2004; 12(3-4):311-319. doi:10.1016/j.trim.2003.12.009.
16. Meyer G A, Farris A L, Sato E, et al. Muscle Progenitor Cell Regenerative Capacity in the Torn Rotator Cuff. J Orthop Res. 2015; (March):421-429. doi:10.1002/jor.22786.
17. Thomas K A, Gibbons M C, Lane J G, Singh A, Ward S R, Engler A J. Rotator cuff tear state modulates self-renewal and differentiation capacity of human skeletal muscle progenitor cells. J Orthop Res. 2016: 12-14. doi:10.1002/jor.23453.
18. Otsuru S, Tamai K, Yamazaki T, Yishikawa H, Kaneda Y. Circulating Bone Marrow-Derived Osteoblast Progenitor Cells Are Recruited to the Bone-Forming Site by the CXCR4/Stromal AND. Stem Cells. 2008; 26:223-234. doi:10.1634/stemcells.2007-0515.
19. Krieger J R, Ogle M E, McFaline-Figueroa J, Segar C E, Temenoff J S, Botchwey E A. Spatially localized recruitment of anti-inflammatory monocytes by SDF-1α-releasing hydrogels enhances microvascular network remodeling. Biomaterials. 2015. doi:10.1016/j.biomaterials.2015.10.045.
20. Kucia M, Jankowski K, Reca R, et al. CXCR4-SDF-1 signalling, locomotion, chemotaxis and adhesion. J Mol Histol. 2004: 233-245.
21. Abbott J D, Huang Y, Liu D, et al. Stromal cell-derived factor-1alpha plays a critical role in stem cell recruitment to the heart after myocardial infarction but is not sufficient to induce homing in the absence of injury. Coron Hear Dis. 2004; 110:3300-3305. doi:10.1161/01.CIR.0000147780.30124.CF.
22. Askari A T, Unzek S, Popovic Z B, et al. Effect of stromal-cell-derived factor 1 on stem-cell homing and tissue regeneration in ischaemic cardiomyopathy. Lancet. 2003; 362:697-703. doi:10.1016/S0140-6736(03)14232-8.
23. Ogle M E, Krieger J R, Tellier L E, McFaline-Figueroa J, Temenoff J S, Botchwey E A. Dual Affinity Heparin-based Hydrogels Achieve Pro-regenerative Immunomodulation and Microvascular Remodeling. ACS Biomater Sci Eng. 2017:acsbiomaterials.6b00706. doi:10.1021/acsbiomaterials.6b00706.
24. Tellier L E, Miller T, McDevitt T C, Temenoff J S. Hydrolysis and sulfation pattern effects on release of bioactive bone morphogenetic protein-2 from heparin-based microparticles. J Mater Chem B. 2015. doi:10.1039/C5TB00933B.
25. Peng Y, Tellier L E, Temenoff J S. Heparin-based hydrogels with tunable sulfation & degradation for anti-inflammatory small molecule delivery. Biomater Sci. 2016; 4(9):1371-1380. doi:10.1039/C6BM00455E.
26. Sadir R, Imberty A, Lortat-jacob H. Heparan Sulfate/Heparin Oligosaccharides Protect Stromal Cell-derived Factor-1 (SDF-1)/CXCL12 against Proteolysis Induced by CD26/Dipeptidyl Peptidase IV. J Biol Chem. 2004; 279(42):43854-43860. doi:10.1074/jbc.M405392200.
27. Purcell B P, Elser J A, Mu A, Margulies K B, Burdick J A. Synergistic effects of SDF-1alpha chemokine and hyaluronic acid release from degradable hydrogels on directing bone marrow derived cell homing to the myocardium. Biomaterials. 2012; 33(31):7849-7857. doi:10.1016/j.biomaterials.2012.07.005.
28. Roy S, Lai H, Zouaoui R, et al. Bioactivity screening of partially desulfated low-molecular-weight heparins: A structure/activity relationship study. Glycobiology. 2011; 21(9):1194-1205. doi:10.1093/glycob/cwr053.
29. Prokoph S, Chavakis E, Levental K R, et al. Sustained delivery of SDF-1α from heparin-based hydrogels to attract circulating pro-angiogenic cells. Biomaterials. 2012; 33(19):4792-4800. doi:10.1016/j.biomaterials.2012.03.039.
30. Anderson E M, Kwee B J, Lewin S A, Raimondo T, Mehta M, Mooney D J. Local Delivery of VEGF and SDF Enhances Endothelial Progenitor Cell Recruitment and Resultant Recovery from Ischemia. Tissue Eng Part A. 2015; 21(7-8):1217-1227. doi:10.1089/ten.tea.2014.0508.
31. Rinker T E, Philbrick B D, Temenoff J S. Core-Shell Microparticles for Protein Sequestration and Controlled Release of a Protein-Laden Core. Acta Biomater. 2016. doi:10.1016/j.actbio.2016.12.042.
32. Hettiaratchi M H, Miller T, Temenoff J S, Guldberg R E, McDevitt T C. Heparin microparticles effects on presentation and bioactivity of bone morphogenetic protein-2. Biomaterials. 2014.
33. Xu X, Jha A K, Duncan R L, Jia X. Heparin-decorated, hyaluronic acid-based hydrogel particles for the controlled release of bone morphogenetic protein 2. Acta Biomater. 2011; 7(8):3050-3059. doi:10.1016/j.actbio.2011.04.018.
34. Hennink W E, van Nostrum C F. Novel crosslinking methods to design hydrogels. Adv Drug Deliv Rev. 2012; 64(SUPPL.):223-236. doi:10.1016/j.addr.2012.09.009.
35. Van De Wetering P, Metters A T, Schoenmakers R G, Hubbell J A. Poly(ethylene glycol) hydrogels formed by conjugate addition with controllable swelling, degradation, and release of pharmaceutically active proteins. J Control Release. 2005; 102(3):619-627. doi:10.1016/j.jconrel.2004.10.029.
36. Seto S P, Miller T, Temenoff J S. Effect of Selective Heparin Desulfation on Preservation of Bone Morphogenetic Protein-2 Bioactivity after Thermal Stress. Bioconjug Chem. 2015; 26(2):286-293. doi:10.1021/bc500565x.
37. Inoue Y, Nagasawa K. Selective N-desulfation of heparin with dimethyl sulfoxide containing water or methanol. Carbohydr Res. 1976; 46:87-95. doi:10.1016/S0008-6215(00)83533-8.
38. Nagasawa K, Inoue Y, Kamata T. Solvolytic Desulfation of Glycosaminoglycuronan Sulfates With Dimethyl Sulfoxide Containing Water or Methanol. Carbohydr Res. 1977; 58:47-55.
39. Liu X, Manzano G, Kim H T, Feeley B T. A Rat Model of Massive Rotator Cuff Tears. J Orthop Res. 2011; (April):588-595. doi:10.1002/jor.21266.
40. Badylak S F, Valentin J E, Ravindra A K, et al. Macrophage phenotype as a determinant of biologic scaffold remodeling. Tissue Eng Part A. 2008; 14(11):1835-1842. doi:10.1089/ten.tea.2007.0264.
41. Kolf C M, Cho E, Tuan R S. Mesenchymal stromal cells. Biology of adult mesenchymal stem cells: regulation of niche, self-renewal and differentiation. Arthritis Res Ther. 2007; 9(1):204. doi:10.1186/ar2116.
42. Kreuger J, Phillipson M. Targeting vascular and leukocyte communication in angiogenesis, inflammation and fibrosis. Nat Rev Drug Discov. 2016; 15(2):125-142. doi:10.1038/nrd.2015.2.
43. Hudalla G A, Eng T S, Murphy W L. An approach to modulate degradation and mesenchymal stem cell behavior in poly(ethylene glycol) networks. Biomacromolecules. 2008; 9(3):842-849. doi:10.1021/bm701179s.
44. Browning M B, Cereceres S N, Luong P T, Cosgriff-Hernandez E M. Determination of the in vivo degradation mechanism of PEGDA hydrogels. J Biomed Res A. 2014; 102(12):4244-4251. doi:10.1002/jbm.a.35096.Determination.
45. Christenson E M, Dadsetan M, Wiggins M, Anderson J M, Hiltner A. Poly (carbonate urethane) and poly (ether urethane) biodegradation: In vivo studies. 2004. doi:10.1002/jbm.a.30002.
46. Xu H, Deng Y, Chen D, Hong W, Lu Y, Dong X. Esterase-catalyzed dePEGylation of pH-sensitive vesicles modified with cleavable PEG-lipid derivatives. 2008; 130:238-245. doi:10.1016/j.jconrel.2008.05.009.
47. Cross D P, Wang C. Stromal-derived factor-1 alpha-loaded PLGA microspheres for stem cell recruitment. Pharm Res. 2011; 28(10):2477-2489. doi:10.1007/s11095-011-0474-x.
48. Zamani M, Prabhakaran M P, Thian E S, Ramakrishna S. Controlled delivery of stromal derived factor-1alpha from poly lactic-co-glycolic acid core-shell particles to recruit mesenchymal stem cells for cardiac regeneration. J Colloid Interface Sci. 2015; 451:144-152. doi:10.1016/j.jcis.2015.04.005.
49. Baumann L, Prokoph S, Gabriel C, Freudenberg U, Werner C, Beck-Sickinger A G. A novel, biased-like SDF-1 derivative acts synergistically with starPEG-based heparin hydrogels and improves eEPC migration in vitro. J Control Release. 2012; 162(1):68-75. doi:10.1016/j.jconrel.2012.04.049.
50. Kimura Y, Tabata Y. Controlled release of stromal-cell-derived factor-1 from gelatin hydrogels enhances angiogenesis. J Biomater Sci Polym Ed. 2010; 21(September 2013):37-51. doi:10.1163/156856209X410193.
51. Misra P, Lebeche D, Ly H, et al. Quantitation of CXCR4 expression in myocardial infarction using 99mTc-labeled SDF-1alpha. J Nucl Med. 2008; 49(6):963-969. doi:10.2967/jnumed.107.050054.
52. Sakiyama-Elbert S E, Hubbell J A. Controlled release of nerve growth factor from a heparin-containing fibrin-based cell ingrowth matrix. J Control Release. 2000; 69(1):149-158. doi:10.1016/50168-3659(00)00296-0.
53. Krieger J R, Tellier L E, Ollukaren M T, Temenoff J S, Botchwey E A. Quantitative Analysis of Immune Cell Subset Infiltration of Supraspinatus Muscle After Severe Rotator Cuff Injury. Regen Eng Transl Med. 2017. doi:10.1007/s40883-017-0030-2.
54. Pizza F X, Peterson J M, Baas J H, Koh T J. Neutrophils contribute to muscle injury and impair its resolution after lengthening contractions in mice. J Physiol. 2005; 562(Pt 3):899-913. doi:10.1113/jphysiol.2004.073965.
55. Arnold L, Henry A, Poron F, et al. Inflammatory monocytes recruited after skeletal muscle injury switch into anti-inflammatory macrophages to support myogenesis. J Exp Med. 2007; 204(5):1057-1069. doi:10.1084/jem.20070075.
56. Sánchez-Martin L, Estecha A, Samaniego R, Sánchez-Ramon S, Vega M Á, Sánchez-Mateos P. The chemokine CXCL12 regulates monocyte-macrophage differentiation and RUNX3 expression. Blood. 2011; 117(1):88-97. doi:10.1182/blood-2009-12-258186.
57. Gupta S K, Pillarisetti K, Lysko P G. Modulation of CXCR4 expression and SDF-1α functional activity during differentiation of human monocytes and macrophages Abstract: Chemoattraction of monocytes by the the regulation of CXCR4 transcription and SDF-1-mRNA in HL-60 cells and does not cause. J Leukoc Biol. 1999; 66(July):135-143.
58. Dezawa M. Bone Marrow Stromal Cells Generate Muscle Cells and Repair Muscle Degeneration. Science (80-). 2005; 309(5732):314-317. doi:10.1126/science.1110364.
59. Kinnaird T, Burnett E S, Shou M, et al. Local Delivery of Marrow-Derived Stromal Cells Augments Collateral Perfusion Through Paracrine Mechanisms. Circulation. 2004; 109(12):1543-1549. doi:10.1161/01.CIR.0000124062.31102.57.
60. Le Blanc K, Mougiakakos D. Multipotent mesenchymal stromal cells and the innate immune system. Nat Rev Immunol. 2012; 12(5):383-396. doi:10.1038/nri3209.
61. Winkler I G, Sims N a, Pettit A R, et al. Bone marrow macrophages maintain hematopoietic stem cell (HSC) niches and their depletion mobilizes HSC Bone marrow macrophages maintain hematopoietic stem cell (HSC) niches and their depletion mobilizes HSC. 2010; 116(23):4815-4829. doi:10.1182/blood-2009-11-253534.
62. Petit I, Jin D, Rafii S. The SDF-1-CXCR4 signaling pathway: a molecular hub modulating neo-angiogenesis. Trends Immunol. 2007; 28(7):299-307.
63. Schirmer S H, van Nooijen F C, Piek J J, van Royen N. Stimulation of collateral artery growth: travelling further down the road to clinical application. Heart. 2009; 95(3):191-197. doi:10.1136/hrt.2007.136119.
64. Van Royen N, Voskuil M, Hoefer I, et al. CD44 Regulates Arteriogenesis in Mice and Is Differentially Expressed in Patients with Poor and Good Collateralization. Circulation. 2004; 109(13):1647-1652. doi:10.1161/01.CIR.0000124066.35200.18.
65. Borselli C, Storrie H, Benesch-Lee F, et al. Functional muscle regeneration with combined delivery of angiogenesis and myogenesis factors. Proc Natl Acad Sci USA. 2010; 107(8):3287-3292. doi:10.1073/pnas.0903875106.
66. Gerwin N, Hops C, Lucke A. Intraarticular drug delivery in osteoarthritis. Adv Drug Deliv Rev. 2006; 58(2):226-242. doi:10.1016/j.addr.2006.01.018.
67. Willett N J, Thote T, Lin A S, et al. Intra-articular injection of micronized dehydrated human amnion/chorion membrane attenuates osteoarthritis development. Arthritis Res Ther. 2014; 16(1):R47. doi:10.1186/ar4476.
68. Goldriing M B. Osteoarthritis and cartilage: the role of cytokines. Curr Rheumatol Rep. 2000; 2(6):459-465. http://www.ncbi.nlm.nih.gov/pubmed/15480070.
69. Pearle A D, Warren R F, Rodeo S A. Basic science of articular cartilage and osteoarthritis. Clin Sports Med. 2005; 24(1):1-12. doi:10.1016/j.csm.2004.08.007.
70. Abramson S B. Inflammation in Osteoarthritis. J Rheumatol. 2004; 31(SUPPL. 70):70-76. doi:10.1097/BOR.0b013e328349c2b1.
71. Kummer J A, Abbink J J, Boer J D E, et al. ANALYSIS OF INTRAARTICULAR FIBRINOLYTIC PATHWAYS IN PATIENTS WITH INFLAMMATORY AND NONINFLAMMATORY JOINT DISEASES. Arthritis Rheum. 1992; 35(8).
72. Brommer P, Dooijewaard G, Dijkmans B, Breedvelf F. Plasminogen activators in synovial fluid and plasma from patients with arthritis. Ann Rheum Dis. 1992; 51:965-968.
73. Belcher C, Fawthrop F, Bunning R, Doherty M. Plasminogen activators and their inhibitors in synovial fluids from normal , osteoarthritis , and rheumatoid arthritis knees. Ann Rheum Dis. 1996; 55:230-236.
74. Ronday H K, Smits H H, Van Muijen G N, et al. Difference in expression of the plasminogen activation system in synovial tissue of patients with rheumatoid arthritis and osteoarthritis. Br J Rheumatol. 1996; 35(5):416-423. http://rheumatology.oxfordjournals.org/content/35/5/416.full.pdf.
75. Busso N, Péclat V, So A, Sappino A P. Plasminogen activation in synovial tissues: differences between normal, osteoarthritis, and rheumatoid arthritis joints. Ann Rheum Dis. 1997; 56(9):550-557. http://www.pubmedcentral.nih.gov/articlerender.fcgi?artid=1752434&tool=pmcentrez&rendertype=abstract.
76. Wisniewski H G, Vilc̆ek J. TSG-6: An IL-1/TNF-inducible protein with anti-inflammatory activity. Cytokine Growth Factor Rev. 1997; 8(2):143-156. doi:10.1016/S1359-6101(97)00008-7.
77. Wisniewski H G, Hua J C, Poppers D M, Naime D, Vilcek J, Cronstein B N. TNF/IL-1-inducible protein TSG-6 potentiates plasmin inhibition by inter-alpha-inhibitor and exerts a strong anti-inflammatory effect in vivo. J Immunol. 1996; 156:1609-1615.
78. Day A J, Drummond S P, Anand S, Bartnik E, Milner C M. A novel chondroprotective property of TSG-6 has therapeutic potential for OA. Osteoarthr Cartil. 2016; 24(2016):519-520. doi:10.1016/j.joca.2016.01.064.
79. Bayliss M T, Howat S L T, Dudhia J, et al. Up-regulation and differential expression of the hyaluronan-binding protein TSG-6 in cartilage and synovium in rheumatoid arthritis and osteoarthritis. Osteoarthr Cartil. 2001; 9(1):42-48. doi:10.1053/joca.2000.0348.
80. Wisniewski H, Colón E, Liublinska V, et al. TSG-6 activity as a novel biomarker of progression in knee osteoarthritis. Osteoarthr Cartil. 2014; 22(2):235-241. doi:10.1016/j.joca.2013.12.004.
81. Chou C, Wisniewski H, Band P, et al. TSG-6 activity reflects severity of inflammation in knee osteoarthritis and acute joint injury. Osteoarthr Cartil. 2016; 24(2016):S81-S82. doi:10.1016/j.joca.2016.01.174.
82. Bárdos T, Kamath R V., Mikecz K, Giant T T. Anti-Inflammatory and Chondroprotective Effect of TSG-6 (Tumor Necrosis Factor-α-Stimulated Gene-6) in Murine Models of Experimental Arthritis. Am J Pathol. 2001; 159(5):1711-1721. doi:10.1016/S0002-9440(10)63018-0.
83. Giant T T, Kamath R V., Bárdos T, et al. Cartilage-specific constitutive expression of TSG-6 protein (product of tumor necrosis factor α-stimulated gene 6) provides a chondroprotective, but not antiinflammatory, effect in antigen-induced arthritis. Arthritis Rheum. 2002; 46(8):2207-2218. doi:10.1002/art.10555.
84. Owen S G, Francis H W, Roberts M S. Disappearance kinetics of solutes from synovial fluid after intra-articular injection. Br J Clin Pharmacol. 1994: 349-355.
Mahoney D J, Mulloy B, Forster M J, et al. Characterization of the interaction between tumor necrosis factor-stimulated gene-6 and heparin: Implications for the inhibition of plasmin in extracellular matrix microenvironments. J Biol Chem. 2005; 280(29):27044-27055. doi:10.1074/jbc.M502068200.
85. Freudenberg U, Zieris A, Chwalek K, et al. Heparin desulfation modulates VEGF release and angiogenesis in diabetic wounds. J Control Release. 2015; 220:79-88. doi:10.1016/j jconrel.2015.10.028.
86. Nakamura S, Ishihara M, Obara K, et al. Controlled release of fibroblast growth factor-2 from an injectable 6-O-desulfated heparin hydrogel and subsequent effect on in vivo vascularization. J Biomed Mater Res A. 2006; 81:364-371. doi:10.1002/jbm.a.
87. Cai S, Liu Y, Zheng Shu X, Prestwich G D. Injectable glycosaminoglycan hydrogels for controlled release of human basic fibroblast growth factor. Biomaterials. 2005; 26(30):6054-6067. doi:10.1016/j.biomaterials 2005.03.012. 88. Tae G, Scatena M, Stayton P S, Hoffman A S. PEG-cross-linked heparin is an affinity hydrogel for sustained release of vascular endothelial growth factor. J Biomater Sci Polym Ed. 2006; 17(1-2):187-197. doi:10.1163/156856206774879090.
89. Zieris A, Dockhorn R, Ro A, et al. Biohybrid Networks of Selectively Desulfated Glycosaminoglycans for Tunable Growth Factor Delivery. Biomacromolecules. 2014; 15:4439-4446.
90. Wood M D, Borschel G H, Sakiyama-elbert S E. Controlled release of glial-derived neurotrophic factor from fibrin matrices containing an affinity-based delivery system. J Biomed Mater Res Part A. 2008: 909-918. doi:10.1002/jbm.a.32043.
91. Baumann H, Scheen H, Huppertz B, Keller R. Novel regio- and stereoselective O-6-desulfation of the glucosamine moiety of heparin with N-methylpyrrolidinone-water or N,N-dimethylformamide-water mixtures. Carbohydr Res. 1998; 308:381-388. doi:10.1016/50008-6215(98)00097-4.
92. Xie L, Lin A S P, Levenston M E, Guldberg R E. Quantitative assessment of articular cartilage morphology via EPIC-??CT. Osteoarthr Cartil. 2009; 17(3):313-320. doi:10.1016/j.joca.2008.07.015.
93. Thote T, Lin A S P, Raji Y, et al. Localized 3D analysis of cartilage composition and morphology in small animal models of joint degeneration. Osteoarthr Cartil. 2013; 21(8):1132-1141. doi:10.1016/j.joca.2013.05.018.
94. Tellier L E, Krieger J R, Sellick A L, et al. Localized SDF-1 Delivery Increases Pro-Healing Bone Marrow-Derived Cells in the Supraspinatus Muscle Following Severe Rotator Cuff Injury. Acta Biomater. 2017: Submitted.
95. Blundell C D, Mahoney D J, Almond A, et al. The link module from ovulation- and inflammation-associated protein TSG-6 changes conformation on hyaluronan binding. J Biol Chem. 2003; 278(49):49261-49270. doi:10.1074/jbc.M309623200.
96. Parkar A A, Day A J. Overlapping sites on the link module of human TSG-6 mediate binding to hyaluronan and chondroitin-4-sulphate. FEBS Lett. 1997; 410(2-3):413-417. doi:10.1016/S0014-5793(97)00621-2.
97. Palmer A W, Guldberg R E, Levenston M E. Analysis of cartilage matrix fixed charge density and three-dimensional morphology via contrast-enhanced microcomputed tomography. Proc Natl Acad Sci U S A. 2006; 103(51):19255-19260. doi:10.1073/pnas.0606406103.
98. Wancket L M, Baragi V, Bove S, Kilgore K, Korytko P J, Guzman R E. Anatomical localization of cartilage degradation markers in a surgically induced rat osteoarthritis model. Toxicol Pathol. 2005; 33(4):484-489. doi:10.1080/01926230590965364.
99. Goldring M B. Chondrogenesis, chondrocyte differentiation, and articular cartilage metabolism in health and osteoarthritis. Ther Adv Musculoskelet Dis. 2012; 4(4):269-285. doi:10.1177/1759720X12448454.
100. Cao T V, La M, Getting S J, Day A J, Perretti M. Inhibitory Effects of TSG-6 Link Module on Leukocyte—Endothelial Cell Interactions In Vitro and In Vivo. 2004; (40):615-624. doi:10.1080/10739680490503438.
101. Nie T, Baldwin A, Yamaguchi N, Kiick K L. Production of Heparin-Functionalized Hydrogels for the Development of Responsive and Controlled Growth Factor Delivery Systems. J Control Release. 2007; 122(3):287-296. doi: 10.1016/j.jconrel.2007.04.019.Production.
102. Choi H, Lee R H, Bazhanov N, Oh J Y, Prockop D J. Anti-inflammatory protein TSG-6 secreted by activated MSCs attenuates zymosan-induced mouse peritonitis by decreasing TLR2/NF-kB signaling in resident macrophages. Immunobiology. 2016; 118(2):330-339. doi:10.1182/blood-2010-12-327353.
103. Wang N, Li Q, Zhang L, et al. Mesenchymal Stem Cells Attenuate Peritoneal Injury through Secretion of TSG-6. PLoS One. 2012; 7(8). doi:10.1371/journal.pone.0043768.
104. Torihashi S, Ho M, Kawakubo Y, et al. Acute and Temporal Expression of TNF-α-stimulated Gene 6 Product,TSG-6,in Mesenchymal Stem Cells Creates Microenvironments Required for Their Successul Transplantation into the Muscle Tissue. J Biol Chem. 2015:jbc.M114.629774. doi:10.1074/jbc.M114.629774.

What is claimed is:

1. A degradable microparticle comprising a GAG-based body and an active agent selected from a protein and a small molecule, wherein the active agent is positively charged.

2. The degradable microparticle of claim 1, wherein the GAG-based body is an N-desulfated heparin-based body.

3. The degradable microparticle of claim 1, wherein the degradable microparticle further comprises DTT or a poly (ethylene glycol)-based linker.

4. The degradable microparticle of claim 3, wherein the poly (ethylene glycol)-based linker is a PEGDA-based linker.

5. The degradable microparticle of claim 1, wherein the active agent is a protein.

6. The degradable microparticle of claim 5, wherein the active agent is SDF-1α or TSG-6.

7. The degradable microparticle of claim 1, wherein the active agent is a small molecule.

8. The degradable microparticle of claim 1, wherein the degradable microparticle is between about 30 and about 80 micrometers in diameter.

9. The degradable microparticle of claim 1, wherein the GAG-based body is an N-desulfated heparin-based body, the degradable microparticle further comprises DTT and a PEGDA-based linker, the active agent is selected from SDF-1α, TSG-6, FTY720 and combinations thereof, and wherein the degradable microparticle is between about 30 and about 80 micrometers in diameter.

10. A pharmaceutical composition comprising a plurality of the degradable microparticles of claim 1 and a pharmaceutically acceptable carrier, additive, or excipient, wherein the pharmaceutical composition is in a liquid injectable dosage form.

11. A method of fabricating a degradable Hep microparticle comprising adding PEGDA and dithiothreitol (DTT) to a solution comprising bovine serum albumin (BSA) in PBS, incubating at 30-40° C. for 20-40 minutes, adding Hep^{-N}MAm to the solution, and incubating the aqueous phase for 20-40 minutes.

12. A method of recruiting pro-regenerative cells to a site of muscle, joint, tendon, or ligament damage, the method comprising introducing into the site of muscle, joint, tendon, or ligament damage a degradable microparticle comprising a GAG-based body and an amount of an active agent selected from a protein and a small molecule effective to recruit pro-regenerative cells to the site of muscle, joint, tendon, or ligament damage.

13. The method of claim 12, wherein the degradable microparticle is introduced into the site of muscle, joint, tendon, or ligament damage by an intramuscular or intra-articular injection.

14. The method of claim 12, wherein a pharmaceutical composition comprising a plurality of the degradable microparticles and a pharmaceutically acceptable carrier, additive, or excipient is introduced into the site of muscle, joint, tendon, or ligament damage by injection.

15. The method of claim 12, wherein the active agent is SDF-1α.

16. The method of claim 12, wherein the muscle damage is rotator cuff damage.

17. The method of claim 12, wherein the degradable microparticle is between about 30 and about 80 micrometers in diameter.

18. A method of reducing inflammation at a site of muscle, joint, tendon, or ligament damage, the method comprising introducing into the site of muscle, joint, tendon, or ligament damage a degradable microparticle comprising a GAG-based body and an amount of an active agent selected from a protein and a small molecule effective to reduce inflammation at the site of muscle, joint, tendon, or ligament damage.

19. The method of claim 18, wherein the joint damage is osteoarthritis and the active agent is TSG-6.

20. The method of claim 18, wherein the degradable microparticle is between about 30 and about 80 micrometers in diameter.
